SEQUENCE LISTING (45 sequences)

SEQ ID NO: 1 - DNA, 426 bp, Mus musculus
  source 1..426: mol_type = other DNA
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaatga

SEQ ID NO: 2 - DNA, 426 bp, Mus musculus
  source 1..426: mol_type = other DNA
atgtggctccagaacctcctcttcctcggtatcgtcgtgtattcactctccgcacctactcgctcacctatcactgtcaccagaccctggaagcacgtggaggccatcaaggaggctctgaacctgctggacgatatgccagtgaccctgaacgaggaggtggaggtggtgagcaacgagttctcctttaagaagctgacctgcgtgcagacaaggctgaagatcttcgagcagggcctgagaggaaactttaccaagctgaagggcgccctgaacatgaccgcttcttactaccagacatactgcccccctacccccgagacagactgtgagacacaggtgaccacatacgccgacttcattgatagcctgaaaacattcctgaccgacattccatttgagtgtaagaagcccgtccagaagtaa

SEQ ID NO: 3 - DNA, 435 bp, Homo sapiens
  source 1..435: mol_type = other DNA
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 4 - DNA, 435 bp, Homo sapiens
  source 1..435: mol_type = other DNA
atgtggctgcagtccctgctgctgctgggcaccgtcgcctgttctatttccgcacccgcaaggtcaccaagtccatctactcagccttgggagcacgtgaacgcaatccaggaggcacggcggctgctgaacctgagccgggacaccgccgccgagatgaacgagacagtggaagtgatcagcgagatgttcgatctgcaggagcccacctgcctgcagacaaggctggagctgtacaagcagggcctgcgcggctctctgaccaagctgaagggcccactgacaatgatggccagccactataagcagcactgcccccctacccccgagacaagctgtgccacccagatcatcacattcgagtcctttaaggagaacctgaaggattttctgctggtcattccatttgattgttgggagcccgtccaggagtaa

SEQ ID NO: 5 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 6 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 7 - DNA, 2010 bp, Gibbon leukemia virus
  source 1..2010: mol_type = other DNA
atggtattgctgcctgggtccatgcttctcacctcaaacctgcaccaccttcggcaccagatgagtcctgggagctggaaaagactgatcatcctcttaagctgcgtattcggcggcggcgggacgagtctgcaaaataagaacccccaccagcccatgaccctcacttggcaggtactgtcccaaactggagacgttgtctgggatacaaaggcagtccagcccccttggacttggtggcccacacttaaacctgatgtatgtgccttggcggctagtcttgagtcctgggatatcccgggaaccgatgtctcgtcctctaaacgagtcagacctccggactcagactatactgccgcttataagcaaatcacctggggagccatagggtgcagctaccctcgggctaggactagaatggcaagctctaccttctacgtatgtccccgggatggccggaccctttcagaagctagaaggtgcggggggctagaatccctatactgtaaagaatgggattgtgagaccacggggaccggttattggctatctaaatcctcaaaagacctcataactgtaaaatgggaccaaaatagcgaatggactcaaaaatttcaacagtgtcaccagaccggctggtgtaacccccttaaaatagatttcacagacaaaggaaaattatccaaggactggataacgggaaaaacctggggattaagattctatgtgtctggacatccaggcgtacagttcaccattcgcttaaaaatcaccaacatgccagctgtggcagtaggtcctgacctcgtccttgtggaacaaggacctcctagaacgtccctcgctctcccacctcctcttcccccaagggaagcgccaccgccatctctccccgactctaactccacagccctggcgactagtgcacaaactcccacggtgagaaaaacaattgttaccctaaacactccgcctcccaccacaggcgacagactttttgatcttgtgcagggggccttcctaaccttaaatgctaccaacccaggggccactgagtcttgctggctttgtttggccatgggccccccttattatgaagcaatagcctcatcaggagaggtcgcctactccaccgaccttgaccggtgccgctgggggacccaaggaaagctcaccctcactgaggtctcaggacacgggttgtgcataggaaaggtgccctttacccatcagcatctctgcaatcagaccctatccatcaattcctccggagaccatcagtatctgctcccctccaaccatagctggtgggcttgcagcactggcctcaccccttgcctctccacctcagtttttaatcagactagagatttctgtatccaggtccagctgattcctcgcatctattactatcctgaagaagttttgttacaggcctatgacaattctcaccccaggactaaaagagaggctgtctcacttaccctagctgttttactggggttgggaatcacggcgggaataggtactggttcaactgccttaattaaaggacctatagacctccagcaaggcctgacaagcctccagatcgccatagatgctgacctccgggccctccaagactcagtcagcaagttagaggactcactgacttccctgtccgaggtagtgctccaaaataggagaggccttgacttgctgtttctaaaagaaggtggcctctgtgcggccctaaaggaagagtgctgtttttacatagaccactcaggtgcagtacgggactccatgaaaaaactcaaagaaaaactggataaaagacagttagagcgccagaaaagccaaaactggtatgaaggatggttcaataactccccttggttcactaccctgctatcaaccatcgctgggcccctattactcctccttctgttgctcatcctcgggccatgcatcatcaataagttagttcaattcatcaatgataggataagtgcagttaaaatttaa

SEQ ID NO: 8 - DNA, 2013 bp, Gibbon leukemia virus
  source 1..2013: mol_type = other DNA
accatggtcctgctgcctgggtctatgctgctgacttctaacctgcaccacctgcgacaccagatgtctcccggctcatggaaacggctgatcatcctgctgagctgcgtgttcggaggaggaggcacctccctgcagaacaagaatcctcaccagccaatgaccctgacatggcaggtgctgtcccagacaggcgacgtggtgtgggataccaaggcagtgcagccaccttggacatggtggcccaccctgaagcctgacgtgtgcgccctggccgcctccctggagtcttgggacatccccggcacagacgtgagcagcagcaagagggtgagaccacccgactctgattatacagccgcctacaagcagatcacctggggcgccatcggctgtagctatcctcgggcccgcacaaggatggccagctccaccttttacgtgtgcccacgcgacggaaggaccctgtctgaggcaaggagatgtggcggcctggagagcctgtattgcaaggagtgggattgtgagaccacaggcacaggctactggctgtctaagtctagcaaggacctgatcaccgtgaagtgggatcagaacagcgagtggacacagaagttccagcagtgccaccagaccggctggtgtaatcccctgaagatcgactttacagataagggcaagctgtccaaggactggatcaccggcaagacatggggcctgagattctacgtgtctggccaccctggcgtgcagtttacaatccggctgaagatcaccaacatgccagcagtggcagtgggaccagacctggtgctggtggagcagggacctccacgcacctccctggccctgccccctccactgccccctagggaggccccaccccctagcctgcccgattctaacagcacagccctggccacctccgcccagacccctacagtgcgcaagaccatcgtgacactgaataccccaccccctaccacaggcgacaggctgttcgatctggtgcagggcgcctttctgacactgaacgccaccaatcctggcgcaaccgagagctgctggctgtgcctggctatgggcccaccctactatgaggcaatcgcctcctctggagaggtggcatattccacagacctggatagatgcagatggggcacccagggcaagctgaccctgacagaggtgtctggccacggcctgtgcatcggcaaggtgccattcacacaccagcacctgtgcaaccagaccctgagcatcaatagctccggcgaccaccagtacctgctgccaagcaaccactcctggtgggcatgctccacaggactgaccccatgtctgagcaccagcgtgttcaaccagaccagagacttttgtatccaggtgcagctgatccctcggatctactattacccagaggaggtgctgctgcaggcctatgataattcccacccaagaacaaagagggaggccgtgtctctgaccctggccgtgctgctgggactgggaatcacagcaggaatcggcacaggcagcaccgccctgatcaagggaccaatcgacctgcagcagggactgacctccctgcagatcgccatcgacgccgatctgagagccctgcaggacagcgtgtccaagctggaggattctctgacctctctgagcgaggtggtgctgcagaacaggaggggcctggacctgctgttcctgaaggagggaggactgtgcgccgccctgaaggaggagtgctgtttttatatcgaccactctggcgccgtgcgggatagcatgaagaagctgaaggagaagctggataagcgccagctggagaggcagaagagccagaattggtacgagggctggttcaacaattccccctggtttaccacactgctgtctaccatcgcaggacctctgttattactgctgctgctgctgatcctgggcccatgtatcatcaacaagctggtgcagtttatcaacgaccgaatctccgcagtgaaaatctaa

SEQ ID NO: 9 - protein, 669 aa, Gibbon leukemia virus
MVLLPGSMLLTSNLHHLRHQMSPGSWKRLIILLSCVFGGGGTSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKREAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINKLVQFINDRISAVKI

SEQ ID NO: 10 - DNA, 759 bp, synthetic construct
  misc_feature 1..759: note = Homo sapiens
  source 1..759: mol_type = other DNA
atgatcgagacctacaatcagacaagcccacggtccgccgcaaccggactgcctatcagcatgaagatcttcatgtacctgctgaccgtgtttctgatcacacagatgatcggctccgccctgttcgccgtgtatctgcacaggagactggacaagatcgaggatgagcgcaatctgcacgaggacttcgtgtttatgaagaccatccagcggtgcaacacaggcgagaggagcctgtctctgctgaattgtgaggagatcaagtcccagttcgagggctttgtgaaggatatcatgctgaacaaggaggagacaaagaaggacgaggatccacagatcgcagcacacgtggtgtccgaggcaaactctaatgccgccagcgtgctgcagtgggccaagaagggctactataccatgaagtctaacctggtgacactggagaatggcaagcagctgaccgtgaagaggcagggcctgtactatatctatgcccaggtgacattctgctctaacagagaggcaagctcccaggcacccttcatcgtgggactgtggctgaagccctctagcggcagcgagaggatcctgctgaaggccgccaatacccactcctctagccagctgtgcgagcagcagtccatccacctgggaggcgtgttcgagctgcagcctggagccagcgtgttcgtgaacgtgacagacccatctcaggtgagccacggcaccggcttcacaagctttggcctgctgaagctgtga

SEQ ID NO: 11 - protein, 252 aa, synthetic construct
  REGION 1..252: note = Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKDEDPQIAAHVVSEANSNAASVLQWAKKGYYTMKSNLVTLENGKQLTVKRQGLYYIYAQVTFCSNREASSQAPFIVGLWLKPSSGSERILLKAANTHSSSQLCEQQSIHLGGVFELQPGASVFVNVTDPSQVSHGTGFTSFGLLKL

SEQ ID NO: 12 - DNA, 1416 bp, Homo sapiens
  source 1..1416: mol_type = other DNA
atgctgccctttctgagcatgctggtgctgctggtgcagcctctgggaaacctgggagccgagatgaagagcctgtcccagagatctgtgcctaacacctgcacactggtcatgtgcagccccaccgagaatggactgcctggaagggacggaagggatggaagggagggccctcggggcgagaagggcgacccaggactgcctggaccaatgggactgagcggactgcagggaccaacaggacctgtgggaccaaagggagagaacggatccgccggagagccaggccctaagggcgagaggggcctgtctggcccccctggcctgccaggcatcccaggccccgccggcaaggagggcccatccggcaagcagggcaatatcggcccccagggcaagcctggcccaaagggcgaggcaggaccaaagggagaagtgggagcacctggcatgcagggatccaccggagcaaagggatctacaggaccaaagggcgagcgcggcgccccaggcgtgcagggcgcccccggcaatgcaggagcagcaggaccagcaggacctgcaggcccacagggcgcccctggctctaggggcccacccggcctgaagggcgacaggggagtgcctggcgataggggcatcaagggagagagcggactgccagattccgccgccctgaggcagcagatggaggccctgaagggcaagctgcagaggctggaggtggccttctcccactaccagaaggccgccctgtttccagacggccacaggagactggacaagatcgaggatgagcgcaacctgcacgaggatttcgtgtttatgaagaccatccagagatgcaacacaggcgagcggtctctgagcctgctgaattgtgaggagatcaagtctcagttcgagggctttgtgaaggacatcatgctgaacaaggaggagaccaagaaggagaatagcttcgagatgcagaagggcgatcagaatccccagatcgcagcacacgtgatcagcgaggcaagctccaagaccacatccgtgctgcagtgggccgagaagggctactataccatgtccaacaatctggtgacactggagaacggcaagcagctgaccgtgaagagacagggcctgtactatatctatgcccaggtgacattctgctctaatcgggaggcctctagccaggccccttttatcgcctctctgtgcctgaagagcccaggcagattcgagcggatcctgctgagggccgccaacacccactcctctgccaagccatgcggacagcagagcatccacctgggaggcgtgttcgagctgcagccaggagcctccgtgtttgtgaatgtgacagacccatcccaggtgtctcacggaaccggcttcacatcctttggcctgctgaagctgtga

SEQ ID NO: 13 - protein, 471 aa, Homo sapiens
MLPFLSMLVLLVQPLGNLGAEMKSLSQRSVPNTCTLVMCSPTENGLPGRDGRDGREGPRGEKGDPGLPGPMGLSGLQGPTGPVGPKGENGSAGEPGPKGERGLSGPPGLPGIPGPAGKEGPSGKQGNIGPQGKPGPKGEAGPKGEVGAPGMQGSTGAKGSTGPKGERGAPGVQGAPGNAGAAGPAGPAGPQGAPGSRGPPGLKGDRGVPGDRGIKGESGLPDSAALRQQMEALKGKLQRLEVAFSHYQKAALFPDGHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 14 - DNA, 1412 bp, Mus musculus
  source 1..1412: mol_type = other DNA
atgctgcccttcctgagcatgctggtgctgctggtgcagcctctgggcaatctgggcgccgagatgaagtccctgtctcagaggagcgtgccaaacacctgcacactggtcatgtgctctccaaccgagaatggactgccaggaagggacggaagagatggaagggagggaccaaggggagagaagggcgaccctggactgcctggaccaatgggactgtccggactgcagggaccaacaggccctgtgggaccaaagggagagaatggaagcgccggagagccaggacctaagggagagaggggcctgtccggcccccctggcctgcctggcatcccaggccccgccggcaaggagggcccttctggcaagcagggcaacatcggaccacagggcaagcctggaccaaagggagaggcaggaccaaagggagaagtgggagcacccggcatgcagggcagcaccggagcaaagggatccaccggccctaagggagagagaggagcacctggagtgcagggcgccccaggcaatgcaggagcagcaggaccagcaggacctgcaggcccacagggcgccccaggcagccggggcccacccggcctgaagggcgacaggggagtgccaggcgataggggcatcaagggagagtccggactgccagactctgccgccctgaggcagcagatggaggccctgaagggcaagctgcagaggctggaggtggccttctcccactaccagaaggccgccctgtttccagacggacacaggagactggataaggtggaggaggaggtgaacctgcacgaggatttcgtgttcatcaagaagctgaagaggtgcaacaagggcgagggcagcctgtccctgctgaattgtgaggagatgcggcgccagttcgaggacctggtgaaggatatcaccctgaacaaggaggagaagaaggagaattcttttgagatgcagaggggcgacgaggatcctcagatcgcagcacacgtggtgtccgaggcaaactctaatgccgccagcgtgctgcagtgggccaagaagggctactataccatgaagtctaacctggtcatgctggagaatggcaagcagctgacagtgaagagagagggcctgtactacgtgtacacccaggtgacattctgcagcaacagagagcccagctcccagcggccttttatcgtgggcctgtggctgaagccctctatcggaagcgagaggatcctgctgaaggcagccaatacccactctagctcccagctgtgcgagcagcagtccgtgcacctgggaggcgtgttcgagctgcaggcaggagcaagcgtgttcgtgaacggacagaggccagccaggtcatccacagagtgggcttctctagctttggcctgctgaagctgtga

SEQ ID NO: 15 - protein, 470 aa, Mus musculus
MLPFLSMLVLLVQPLGNLGAEMKSLSQRSVPNTCTLVMCSPTENGLPGRDGRDGREGPRGEKGDPGLPGPMGLSGLQGPTGPVGPKGENGSAGEPGPKGERGLSGPPGLPGIPGPAGKEGPSGKQGNIGPQGKPGPKGEAGPKGEVGAPGMQGSTGAKGSTGPKGERGAPGVQGAPGNAGAAGPAGPAGPQGAPGSRGPPGLKGDRGVPGDRGIKGESGLPDSAALRQQMEALKGKLQRLEVAFSHYQKAALFPDGHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSIGSERILLKAANTHSSSQLCEQQSVHLGGVFELQAGASVFVNVTEASQVIHRVGFSSFGLLKL

SEQ ID NO: 16 - DNA, 786 bp, Homo sapiens
  source 1..786: mol_type = other DNA
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 17 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 18 - DNA, 783 bp, Mus musculus
  source 1..783: mol_type = other DNA
atgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctga

SEQ ID NO: 19 - protein, 260 aa, Mus musculus
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 20 - DNA, 930 bp, Mus musculus
  source 1..930: mol_type = other DNA
atggatcagcacacactggacgtggaggataccgctgacgctaggcacccagctggcacctcctgcccttctgatgccgctctgctgcgcgacacaggactgctggccgatgccgctctgctgtctgacacagtgcggccaaccaacgccgctctgccaaccgatgctgcttaccctgctgtgaacgtgagggacagagaggctgcttggccacctgccctgaacttctgcagccgccaccctaagctgtacggcctggtggccctggtgctgctgctgctgatcgctgcttgcgtgccaatctttacccggacagagccacgccccgctctgacaatcaccacatcccccaacctgggcaccagggagaacaacgccgatcaggtgacaccagtgtctcacatcggctgccccaacaccacacagcagggaagcccagtgttcgccaagctgctggctaagaaccaggccagcctgtgcaacaccacactgaactggcacagccaggacggagctggaagctcctacctgtcccagggcctgagatacgaggaggataagaaggagctggtggtggactcccctggactgtactacgtgttcctggagctgaagctgtctccaacctttacaaacaccggccacaaggtgcagggatgggtgtctctggtgctgcaggctaagccccaggtggacgatttcgataacctggccctgaccgtggagctgtttccttgtagcatggagaacaagctggtggacaggtcttggagccagctgctgctgctgaaggctggccacaggctgtccgtgggactgagagcctacctgcacggcgcccaggatgcttacagagactgggagctgagctaccctaacaccacatccttcggactgtttctggtgaagcctgacaacccatgggagtga

SEQ ID NO: 21 - DNA, 765 bp, Homo sapiens
  source 1..765: mol_type = other DNA
atggagtacgcctctgacgccagcctggatccagaggccccttggccacctgcaccaagggcccgcgcctgccgcgtgctgccctgggccctggtggccggcctgttattactgctgctgctggccgccgcctgcgccgtgttcctggcatgtccttgggccgtgagcggagccagagcctccccaggctctgccgccagccctcggctgagagagggaccagagctgtccccagacgatccagcaggcctgctggacctgaggcagggaatgtttgcccagctggtggcccagaacgtgctgctgatcgacggccccctgtcctggtactctgatcctggcctggccggcgtgtctctgaccggcggcctgagctataaggaggatacaaaggagctggtggtggccaaggccggcgtgtactacgtgttcttccagctggagctgaggagagtggtggcaggagagggctctggaagcgtgtccctggccctgcacctgcagcccctgcggagcgccgcaggagccgccgccctggccctgaccgtggacctgccaccagccagctccgaggcaaggaattccgccttcggctttcagggcagactgctgcacctgtctgccggacagaggctgggagtgcacctgcacaccgaggccagggcccgccacgcatggcagctgacccagggagcaacagtgctgggcctgttccgcgtgacacctgagatcccagcaggcctgcctagcccacggtccgagtga

SEQ ID NO: 22 - DNA, 1389 bp, Mus musculus
  source 1..1389: mol_type = other DNA
atgctgcctttcctgtccatgctggtgctgctggtgcagccactgggcaacctgggagccgagatgaagtctctgagccagcgcagcgtgcctaacacctgcacactggtcatgtgctcccctacagagaacggcctgccaggaagggacggaagagatggaagggagggaccaaggggagagaagggcgaccccggactgcctggaccaatgggactgagcggcctgcagggaccaaccggccccgtgggacctaagggagagaacggatccgctggagagccaggacctaagggagagagaggactgtctggaccacctggactgccaggaatcccaggaccagctggcaaggagggaccatccggcaagcagggaaacatcggaccacagggaaagcctggaccaaagggagaggctggacctaagggagaagtgggcgccccaggaatgcagggctctacaggagctaagggcagcaccggaccaaagggagagaggggagcccccggagtgcagggagcccctggcaacgctggagccgctggcccagccggacccgctggccctcagggagcccccggctctaggggaccaccaggcctgaagggagacagaggcgtgcccggagatcggggcatcaagggagagagcggcctgcctgactccgccgctctgagacagcagatggaggctctgaagggcaagctgcagcggctggaggtggccttctcccactaccagaaggccgctctgtttcctgacggaaggacagagcccaggcctgctctgaccatcaccacatctccaaacctgggcacaagagagaacaacgccgatcaggtgacccccgtgtctcacatcggatgccctaacaccacacagcagggcagccccgtgtttgccaagctgctggctaagaaccaggccagcctgtgcaacaccacactgaactggcactcccaggatggcgccggaagctcctacctgtctcagggcctgcggtacgaggaggacaagaaggagctggtggtggatagcccaggcctgtactacgtgttcctggagctgaagctgtcccccacctttacaaacaccggacacaaggtgcagggatgggtgagcctggtgctgcaggctaagccccaggtggacgatttcgacaacctggccctgaccgtggagctgtttccttgctctatggagaacaagctggtggatagatcctggagccagctgctgctgctgaaggctggacaccgcctgagcgtgggcctgagggcttacctgcacggagctcaggacgcttacagggattgggagctgtcctaccctaacaccacatctttcggcctgtttctggtgaagccagacaacccctgggagtga

SEQ ID NO: 23 - DNA, 1389 bp, Homo sapiens
  source 1..1389: mol_type = other DNA
atgctgctgttcctgctgtccgccctggtgctgctgacccagcctctgggctacctggaggccgagatgaagacctattctcaccggacaatgccaagcgcctgcacactggtcatgtgcagcagcgtggagtctggcctgccaggaagggacggaagggatggaagggagggacctagaggcgagaagggcgaccctggcctgccaggagcagcaggacaggcaggaatgcccggccaggccggccccgtgggacctaagggcgacaacggaagcgtgggagagccaggaccaaagggcgataccggcccttccggaccacctggaccaccaggcgtgcctggcccagccggcagggagggccctctgggcaagcagggcaatatcggcccacagggcaagcccggccctaagggcgaggccggccccaagggcgaagtgggcgcccctggcatgcagggaagcgccggagcccgcggcctggccggacctaagggcgagagaggcgtgcctggagagaggggcgtgccaggaaacacaggcgcagcaggatctgccggagcaatgggaccccagggcagccctggcgccaggggccctccaggcctgaagggcgacaagggcatcccaggcgataagggagcaaagggagagagcggcctgccagatgtggcctccctgcgccagcaggtggaggccctgcagggccaggtgcagcacctgcaggccgccttctctcagtacaagaaggtggagctgtttccaaacggcgcctgcccctgggccgtgagcggagcccgggcctccccaggctctgccgccagccctaggctgcgcgagggaccagagctgagcccagacgatccagcaggcctgctggacctgagacagggaatgttcgcccagctggtggcccagaatgtgctgctgatcgacggcccactgtcctggtactctgatccaggcctggccggcgtgtccctgaccggcggcctgtcttataaggaggatacaaaggagctggtggtggccaaggccggcgtgtactacgtgttcttccagctggagctgaggagagtggtggcaggagagggatccggatctgtgagcctggccctgcacctgcagcccctgcggtccgccgcaggagccgccgccctggccctgaccgtggacctgccacctgcctctagcgaggcacgcaattccgccttcggctttcagggccggctgctgcacctgtctgccggacagagactgggagtgcacctgcacaccgaggcccgggccagacacgcctggcagctgacccagggagcaacagtgctgggcctgtttagggtgacacctgagatcccagccggcctgccaagcccccgctccgagtga

SEQ ID NO: 24 - DNA, 522 bp, Mus musculus
  source 1..522: mol_type = other DNA
atggaggagatgcctctgagggagagctccccacagagggccgagagatgcaagaagagctggctgctgtgcatcgtggctctgctgctgatgctgctgtgctctctgggcaccctgatctacacaagcctgaagccaaccgccatcgagtcctgtatggtgaagttcgagctgtctagctccaagtggcacatgacatcccccaagcctcactgcgtgaacaccacatctgacggaaagctgaagatcctgcagagcggcacctacctgatctacggacaggtcatccccgtggacaagaagtacatcaaggataacgcccctttcgtggtgcagatctacaagaagaacgacgtgctgcagacactgatgaacgattttcagatcctgcccatcggcggagtgtacgagctgcacgctggcgacaacatctacctgaagttcaactccaaggatcacatccagaagaccaacacatactggggaatcatcctgatgccagatctgccctttatctcttga

SEQ ID NO: 25 - DNA, 600 bp, Homo sapiens
  source 1..600: mol_type = other DNA
atgaccctgcacccaagccccatcacatgcgagttcctgttttctaccgccctgatcagcccaaagatgtgcctgagccacctggagaatatgcccctgtcccactctcggacacagggagcccagagaagctcctggaagctgtggctgttctgctctatcgtgatgctgctgttcctgtgcagcttttcctggctgatcttcatctttctgcagctggagacagccaaggagccttgcatggccaagtttggccctctgccatccaagtggcagatggcctctagcgagcccccttgcgtgaacaaggtgagcgactggaagctggagatcctgcagaacggcctgtacctgatctatggccaggtggcccccaacgccaattacaacgacgtggcccctttcgaggtgcggctgtataagaacaaggatatgatccagaccctgacaaataagtctaagatccagaacgtgggcggcacatacgagctgcacgtgggcgacaccatcgacctgatcttcaacagcgagcaccaggtgctgaagaacaatacatattggggcatcatcctgctggccaacccccagtttatctcctga

SEQ ID NO: 26 - DNA, 1164 bp, Mus musculus
  source 1..1164: mol_type = other DNA
atgctgcctttcctgtctatgctggtgctgctggtgcagccactgggcaacctgggagccgagatgaagagcctgtcccagagatccgtgcccaacacctgcacactggtcatgtgctctcctaccgagaacggcctgccaggaagggacggaagagatggaagggagggacctcggggagagaagggcgacccaggactgcctggaccaatgggactgagcggcctgcagggaccaacaggccccgtgggacctaagggagagaacggaagcgccggagagccaggacctaagggagagaggggactgtccggaccacctggactgcctggaatcccaggaccagctggcaaggagggaccatccggcaagcagggaaacatcggaccacagggaaagcctggaccaaagggagaggctggaccaaagggagaagtgggcgctcctggaatgcagggctccaccggagccaagggctctacaggaccaaaaggagagaggggagctcccggagtgcagggagcccctggcaacgctggagccgctggcccagccggacccgctggccctcagggagccccaggcagcaggggaccacccggcctgaagggcgacaggggcgtgccaggagataggggcatcaagggagagtctggcctgccagacagcgccgctctgagacagcagatggaggccctgaagggcaagctgcagcggctggaggtggctttctcccactaccagaaggccgctctgtttccagatggcagcctgaagcccaccgccatcgagtcctgcatggtgaagtttgagctgagctcctctaagtggcacatgacatctcccaagcctcactgcgtgaacaccacatctgacggcaagctgaagatcctgcagagcggcacctacctgatctacggccaggtcatccccgtggacaagaagtacatcaaggataacgcccctttcgtggtgcagatctacaagaagaacgacgtgctgcagacactgatgaacgattttcagatcctgccaatcggcggagtgtacgagctgcacgctggcgacaacatctacctgaagttcaactctaaggatcacatccagaagaccaacacatactggggcatcatcctgatgccagatctgccctttatcagctga

SEQ ID NO: 27 - DNA, 1152 bp, Homo sapiens
  source 1..1152: mol_type = other DNA
atgctgctgttcctgctgtctgccctggtgctgctgacccagccactgggctacctggaggccgagatgaagacctattcccaccgcacaatgccttctgcctgcacactggtcatgtgcagcagcgtggagagcggcctgccaggaagggacggaagagatggaagggagggacccagaggcgagaagggcgaccctggcctgccaggagcagcaggacaggcaggaatgccaggccaggccggccccgtgggccctaagggcgacaatggatccgtgggagagccaggaccaaagggcgataccggcccttctggaccacctggaccaccaggcgtgcctggaccagcaggaagagagggacctctgggcaagcagggaaacatcggaccacagggcaagccaggccctaagggcgaggccggccccaagggcgaagtgggcgcccctggcatgcagggatccgccggagccaggggcctggccggacctaagggcgagcgcggcgtgcctggagagaggggcgtgccaggaaatacaggcgcagcaggatctgccggagcaatgggaccacagggcagccccggcgccagaggccctccaggcctgaagggcgacaagggaatccctggcgataagggagcaaagggagagagcggcctgccagacgtggcctccctgaggcagcaggtggaggccctgcagggacaggtgcagcacctgcaggccgccttcagccagtacaagaaggtggagctgtttccaaatggcgagacagccaaggagccctgcatggccaagttcggcccactgcccagcaagtggcagatggcctctagcgagcccccttgcgtgaacaaggtgagcgattggaagctggagatcctgcagaacggcctgtacctgatctatggccaggtggcccccaacgccaattacaacgacgtggccccttttgaggtgcggctgtataagaacaaggatatgatccagaccctgacaaataagtctaagatccagaacgtgggaggcacctacgagctgcacgtgggcgacacaatcgacctgatcttcaacagcgagcaccaggtgctgaagaacaatacatattggggcatcatcctgctggccaacccccagtttatctcctga

SEQ ID NO: 28 - DNA, 597 bp, Mus musculus
  source 1..597: mol_type = other DNA
atggagggcgagggagtgcagcccctggatgagaacctggagaacggctcccggcctcgcttcaagtggaagaagaccctgcggctggtggtgtctggaatcaagggcgccggaatgctgctgtgctttatctacgtgtgcctgcagctgagctcctctcccgccaaggatccccctatccagaggctgagaggagctgtgaccaggtgcgaggacggacagctgttcatcagctcctacaagaacgagtaccagacaatggaggtgcagaacaacagcgtggtcatcaagtgtgatggcctgtacatcatctacctgaagggatccttctttcaggaggtgaagatcgacctgcactttcgggaggatcacaacccaatctctatccccatgctgaacgacggcaggagaatcgtgttcacagtggtggccagcctggcttttaaggacaaggtgtacctgaccgtgaacgccccagatacactgtgcgagcacctgcagatcaacgacggagagctgatcgtggtgcagctgacccctggctactgtgctccagagggatcttaccacagcacagtgaaccaggtgcccctgtga

SEQ ID NO: 29 - DNA, 552 bp, Homo sapiens
  source 1..552: mol_type = other DNA
atggagagggtgcagcccctggaggagaacgtgggaaatgccgcccggcctagattcgagaggaacaagctgctgctggtggcctctgtgatccagggcctgggcctgctgctgtgcttcacctacatctgtctgcacttttctgccctgcaggtgagccacagatacccccgcatccagagcatcaaggtgcagttcaccgagtataagaaggagaagggctttatcctgacatcccagaaggaggacgagatcatgaaggtgcagaacaattctgtgatcatcaactgcgatggcttctacctgatctccctgaagggctatttttctcaggaagtgaatatcagcctgcactatcagaaggacgaggagccactgtttcagctgaagaaggtgcggagcgtgaattccctgatggtggccagcctgacctacaaggacaaggtgtatctgaacgtgaccacagataatacatccctggacgatttccacgtgaacggcggcgagctgatcctgatccaccagaatcccggcgagttttgcgtgctgtga

SEQ ID NO: 30 - DNA, 1215 bp, Mus musculus
  source 1..1215: mol_type = other DNA
atgctgcccttcctgtccatgctggtgctgctggtgcagcctctgggcaacctgggagccgagatgaagtctctgagccagagatccgtgccaaacacctgcacactggtcatgtgctctcccaccgagaacggcctgcctggaagggacggaagagatggaagggagggaccccggggagagaagggcgatcctggactgccaggacctatgggactgagcggcctgcagggaccaacaggccccgtgggacctaagggagagaacggaagcgccggagagccaggaccaaagggagagaggggactgtccggcccacctggactgcctggaatccctggaccagctggcaaggagggaccttccggcaagcagggaaacatcggaccacagggaaagccaggacctaagggagaggctggaccaaagggagaagtgggcgctcccggaatgcagggctctaccggagccaagggcagcacaggacctaagggagagaggggagctccaggagtgcagggagcccccggcaacgctggagctgctggaccagctggaccagctggccctcagggagccccaggctctaggggaccaccaggcctgaagggcgacaggggcgtgccaggagataggggcatcaagggagagagcggcctgccagattccgccgctctgagacagcagatggaggccctgaagggcaagctgcagcggctggaggtggctttcagccactaccagaaggccgctctgtttcctgacggcagctcctctccagccaaggatcctccaatccagcggctgcgcggagctgtgaccaggtgcgaggatggccagctgttcatcagctcctacaagaacgagtaccagacaatggaggtgcagaacaactctgtggtcatcaagtgtgacggcctgtacatcatctacctgaagggcagcttctttcaggaggtgaagatcgacctgcactttagagaggatcacaacccaatctccatccccatgctgaacgacggcaggagaatcgtgttcaccgtggtggcctctctggcttttaaggacaaggtgtacctgaccgtgaacgcccccgatacactgtgcgagcacctgcagatcaacgacggcgagctgatcgtggtgcagctgacccctggatactgtgctccagagggctcctaccactctacagtgaaccaggtgcctctgtga

SEQ ID NO: 31 - DNA, 1170 bp, Homo sapiens
  source 1..1170: mol_type = other DNA
atgctgctgttcctgctgagcgccctggtgctgctgacccagccactgggctacctggaggccgagatgaagacctattcccacagaacaatgccttctgcctgcacactggtcatgtgcagcagcgtggagtccggcctgccaggaagggacggcagagatggcagggagggccccaggggcgagaagggcgaccccggcctgcctggagcagcaggccaggccggcatgccaggccaggccggcccagtgggccccaagggcgacaacggcagcgtgggcgagcccggccctaagggcgataccggcccctccggcccccctggcccacccggcgtgccaggaccagcaggaagggagggaccactgggcaagcagggcaatatcggacctcagggcaagcctggaccaaagggagaggcaggaccaaagggagaagtgggcgcccctggcatgcagggatctgccggagcccggggcctggccggccccaagggcgagagaggcgtgcccggcgagaggggcgtgcctggcaacacaggcgccgccggctccgccggcgccatgggacctcagggctctccaggagccagaggccctccaggcctgaagggcgacaagggaatccctggcgataagggagcaaagggagagagcggcctgccagacgtggcctccctgcggcagcaggtggaggccctgcagggccaggtgcagcacctgcaggccgccttcagccagtacaagaaggtggagctgtttcctaatggcgtgtctcaccgctacccacggatccagagcatcaaggtgcagttcaccgagtataagaaggagaagggctttatcctgacatctcagaaggaggacgagatcatgaaggtgcagaacaatagcgtgatcatcaactgcgatggcttctacctgatcagcctgaagggctatttttcccaggaagtgaatatctctctgcactatcagaaggatgaggagcctctgtttcagctgaagaaggtgagatctgtgaacagcctgatggtggcctccctgacctacaaggacaaggtgtatctgaacgtgaccacagataatacatctctggacgatttccacgtgaacggcggcgagctgatcctgatccaccagaatcccggcgagttttgcgtgctgtga

SEQ ID NO: 32 - DNA, 969 bp, Mus musculus
  source 1..969: mol_type = other DNA
atgcagctgaagtgtccatgcttcgtgtccctgggaacaagacagcccgtctggaagaaactgcacgtgagctccggcttctttagcggcctggggctgtttctgctgctgctgtctagtctgtgcgccgcttccgcagagactgaagtcggagccatggtgggcagtaacgtggtcctgtcatgcatcgacccacaccgacggcatttcaacctgtctggcctgtacgtgtattggcagattgagaatcccgaagtgtcagtcacctactatctgccttacaagagcccagggatcaacgtggactcaagctataaaaatagggggcacctgtccctggattctatgaagcagggaaacttcagcctgtacctgaaaaatgtgacccctcaggacacacaggagttcacttgtcgcgtctttatgaacactgcaaccgaactggtgaagattctggaggaagtggtccggctgagagtcgcagccaactttagcactcctgtgatctctaccagtgattcctctaatccaggccaggagcggacatatacttgcatgtctaagaacggataccccgaacctaatctgtattggatcaacaccacagacaatagtctgattgataccgctctgcagaacaatacagtctacctgaacaagctggggctgtatgacgtgatctctactctgcggctgccatggaccagtagaggagatgtgctgtgctgcgtggagaacgtggccctgcaccagaatatcacctcaattagccaggctgagtcctttaccggcaacaatacaaagaatcctcaggagacacataacaatgaactgaaagtgctggtgccagtgctggccgtcctggctgcagcagctttcgtgtcttttatcatctacagaaggacccgccctcaccgctcatacactggacctaagaccgtgcagctggaactgacagaccatgcttga

SEQ ID NO: 33 - DNA, 909 bp, Homo sapiens
  source 1..909: mol_type = other DNA
atgcgtctgggttcacctggtctgctgtttctgctgttttcaagtctgcgtgctgatactcaggagaaggaagtccgggctatggtcggaagtgacgtggagctgtcatgcgcttgtcccgaagggtcccggttcgacctgaacgatgtctacgtgtattggcagacctctgagagtaagaccgtggtcacataccacatccctcagaactccagcctggaaaatgtggattcaaggtatcggaacagagccctgatgtcccctgctggcatgctgcggggagacttctctctgagactgtttaatgtgacaccacaggatgagcagaaattccattgcctggtcctgtcacagtccctgggatttcaggaggtgctgagtgtcgaagtgactctgcacgtcgccgctaatttctccgtgcctgtggtcagcgcaccacatagcccctctcaggacgagctgacctttacatgtacttccatcaacggctacccccgccctaacgtgtactggattaacaagactgacaatagcctgctggatcaggcactgcagaacgacaccgtgtttctgaatatgcgaggactgtacgatgtggtcagcgtcctgcgtattgccaggaccccatctgtgaacatcgggtgctgtattgagaacgtcctgctgcagcagaatctgacagtggggagccagactggtaatgacatcggcgagagggataagattaccgaaaaccccgtgagtacaggcgagaagaacgcagccacatggtcaatcctggctgtgctgtgcctgctggtggtcgtggctgtcgcaattggctgggtgtgccgcgatcggtgtctgcagcactcttatgccggtgcttgggcagtgagtccagagactgaactgaccggccatgtctaa

SEQ ID NO: 34 - DNA, 1574 bp, Mus musculus
  source 1..1574: mol_type = other DNA
cttaagatggaaactgatactctgctgctctgggtgctgctcctctgggtgcctggttcaactggggacattcgacgggctgacattgtgatgacccagaccacactgagcctgcccgtgtccctgggcgaccaggccagcatctcctgccggagctcccagtctatcgtgcacagcaacggaaacacatacctggagtggtatctgcagaagcctggccagtccccaaagctgctgatctacaaggtgtccaacaggttcagcggcgtgcctgaccgcttttctggaagcggctccggaacagatttcaccctgaagatcagcagggtggaggctgaggacctgggcgtgtactactgcttccagggatcccacgtgccttacacctttggcggaggcacaaagctggagatcaagagagccgatgctgctccaaccgtgtctggaagcggaggcgggggttctggaggcggtgggagcggtggcggagggtctgaggctaagctgcaggagagcggccccgtgctggtgaagcctggagccagcgtgaagatgtcctgtaaggcttctggatacaccttcacagactactacatgaactgggtgaagcagagccacggcaagtccctggagtggatcggagtgatcaacccttacaacggcgacacctcttacaaccagaagtttaagggcaaggccaccctgacagtggataagtctagctccaccgcttacatggagctgaacagcctgacatccgaggattctgccgtgtactactgtgctaggtactacggaagctggttcgcctactggggccagggaacactgatcaccgtgtccacagccaagaccacaccccctagcgtgtaccccctggctcctaggtctagcagaggctgcaagccatgcatctgtaccgtgcccgaggtgagcagcgtgttcatctttccacccaagcccaaggacgtgctgaccatcacactgacccctaaggtgacatgcgtggtggtggatatcagcaaggacgatccagaggtgcagttctcctggtttgtggacgatgtggaggtgcacaccgcccagacacagccaagggaggagcagttcaactccacctttagatccgtgtctgagctgcccatcatgcaccaggactggctgaacggaaaggagttcaagtgccgggtgaactccgccgcttttcctgctccaatcgagaagaccatctctaagacaaagggccgcccaaaggctccacaggtgtacaccatccctccacccaaggagcagatggctaaggataaggtgagcctgacctgtatgatcacagacttctttcccgaggatatcacagtggagtggcagtggaacggacagcctgccgagaactacaagaacacccagccaatcatggacacagatggctcttacttcgtgtacagcaagctgaacgtgcagaagtctaactgggaggctggcaacaccttcacctgcagcgtgctgcacgaaggtctccataatcaccacaccgaaaagagcctcagtcacagccctgggaaatgaggcgcgcc

SEQ ID NO: 35 - DNA, 1484 bp, Homo sapiens
  source 1..1484: mol_type = other DNA
cttaagatggaaactgacaccctgctgctgtgggtcctgctgctgtgggtgcctggatccaccggcgatatcgtgctgacccagtctcctggcacactgagtctgtcaccaggggagcgagcaacactgtcttgtagagccagccagtctgtgggaagctcctacctggcttggtatcagcagaagccaggccaggcacccaggctgctgatctacggagccttcagccgggccactggcattccagacaggttctctggaagtggctcagggaccgacttcaccctgaccatcagccgactggagcccgaagacttcgccgtgtactattgccagcagtacggctctagtccttggacttttggacagggcaccaaagtggagatcaagcgcggcgggggaggctctgggggaggcgggagtggaggcgggggatcacaggtccagctggtggaaagcggcgggggagtggtccagccaggccggagcctgcggctgagctgcgccgcttcaggattcacattttcaagctataccatgcactgggtccggcaggcaccagggaagggactggagtgggtgaccttcatcagctatgacggcaacaacaagtattacgctgattccgtgaaagggaggtttaccattagccgcgacaactccaaaaatacactgtacctgcagatgaacagcctgcgggccgaggatactgctatctactattgcgcaagaaccgggtggctgggacccttcgactattggggccaggggactctggtcaccgtgtcctctgataagacacacacatgccctccctgtcctgcaccagagctgctgggcgggccatccgtgttcctgtttccacccaagcctaaagacaccctgatgatcagccggacacctgaagtcacttgcgtggtcgtggacgtgagtcacgaggatccagaagtcaagtttaactggtacgtggatggcgtcgaggtgcataatgccaagaccaaacctcgcgaggaacagtacaatagcacatatcgagtcgtgtccgtcctgactgtgctgcatcaggattggctgaacggcaaagagtataagtgcaaagtgagcaataaggcactgcctgccccaatcgagaaaacaatttccaaggctaaaggccagcccagggaacctcaggtgtacactctgcctccaagtcgcgaggaaatgaccaagaaccaggtgagcctgacctgtctggtgaaagggttctatccatcagacattgcagtggagtgggaaagcaatggacagcccgaaaacaattacaagaccacaccccctgtgctggacagcgatggctccttctttctgtattctaagctgactgtggataaaagtcgctggcagcaggggaacgtctttagctgttccgtgatgcatgaggctctgcacaatcattacacacagaagtctctgagtctgtcacccggcaaatgaggcgcgcc

SEQ ID NO: 36 - DNA, 632 bp, synthetic construct
  misc_feature 1..632: note = CMV promoter
  source 1..632: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaatt

SEQ ID NO: 37 - DNA, 394 bp, synthetic construct
  misc_feature 1..394: note = RSV promoter
  source 1..394: mol_type = other DNA
tgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctcc

SEQ ID NO: 38 - DNA, 188 bp, synthetic construct
  misc_feature 1..188: note = BGH polyA
  source 1..188: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagac

SEQ ID NO: 39 - DNA, 249 bp, synthetic construct
  misc_feature 1..249: note = SV40 late polyA
  source 1..249: mol_type = other DNA
gacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 40 - DNA, 345 bp, synthetic construct
  misc_feature 1..345: note = SV40 enhancer promoter
  source 1..345: mol_type = other DNA
gctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagct

SEQ ID NO: 41 - DNA, 99 bp, synthetic construct
  misc_feature 1..99: note = Rabbit beta-globin polyA
  source 1..99: mol_type = other DNA
gacctctggctaataaaggaaatttattttcattgcaatagtgtgttggaattttttgtgtctctcactcggaaggacatatgggagggcaaatcattt

SEQ ID NO: 42 - DNA, 723 bp, synthetic construct
  misc_feature 1..723: note = GFP
  source 1..723: mol_type = other DNA
accatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 43 - DNA, 454 bp, synthetic construct
  misc_feature 1..454: note = MoMuLV LTR
  source 1..454: mol_type = other DNA
ttaattaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgcttaag

SEQ ID NO: 44 - DNA, 1349 bp, synthetic construct
  misc_feature 1..1349: note = EF1alpha promoter
  source 1..1349: mol_type = other DNA
ttaattaagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcggttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacttaag

SEQ ID NO: 45 - DNA, 481 bp, synthetic construct
  misc_feature 1..481: note = HGH polyA
  source 1..481: mol_type = other DNA
gacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttt

(19) United States
(12) Patent Application Publication
(10) Pub. No.: US 2025/0352646 A1
(43) Pub. Date: Nov. 20, 2025

(54) ENGINEERED VIRUS

(71) Applicant: Replimune Limited, Oxfordshire (GB)

(72) Inventor: Robert Stuart Coffin, London (GB)

(73) Assignee: Replimune Limited, Oxfordshire (GB)

(21) Appl. No.: 19/277,600

(22) Filed: Jul. 23, 2025

Related U.S. Application Data
(63) Continuation of application No. 18/163,837, filed on Feb. 2, 2023, now Pat. No. 12,397,053.
Application No. 18/163,837 is a continuation of application No. 17/164,635, filed on Feb. 1, 2021, now Pat. No. 12,049,647.
Application No. 17/164,635 is a continuation of application No. 16/068,830, filed on Jul. 9, 2018, now Pat. No. 10,947,513.

(30) Foreign Application Priority Data
Jan. 8, 2016 (GB) 1600380.8
Jan. 8, 2016 (GB) 1600381.6
Jan. 8, 2016 (GB) 1600382.4

(51) Int. Cl. A61K 39/395; A61K 35/763; A61K 39/00; A61K 45/06; A61P 35/00; C07K 14/005; C07K 14/535; C07K 16/28; C12N 7/00

(52) U.S. Cl. CPC A61K 39/39558; A61K 35/763; A61K 39/3955; A61K 45/06; A61P 35/00; C07K 14/005; C07K 14/535; C07K 16/2818; C12N 7/00; A61K 2039/505; C12N 2710/16621; C12N 2710/16622; C12N 2710/16632; C12N 2710/16633; C12N 2710/16643; C12N 2740/13022

(57) ABSTRACT

The present invention relates to oncolytic virus comprising: (i) a GM-CSF-encoding gene; and (ii) an immune co-stimulatory pathway activating molecule or an immune co-stimulatory pathway activating molecule-encoding gene.

### CROSS-REFERENCE TO RELATED APPLICATIONS

This application is a continuation of U.S. Non-Provisional patent application Ser. No. 18/163,837 filed Feb. 2, 2023, which is a continuation of U.S. Non-Provisional patent application Ser. No. 17/164,635 filed Feb. 1, 2021, now U.S. Pat. No. 12,049,647 granted Jul. 30, 2024, which is a continuation of U.S. Non-Provisional patent application Ser. No. 16/068,830 filed Jul. 9, 2018, now U.S. Pat. No. 10,947,513 issued Mar. 16, 2021, which is a national phase application under 35 U.S.C. § 371 to International Application No. PCT/GB2017/050038 filed Jan. 9, 2017, which claims the benefit of priority to Great Britain Patent Application Serial Nos. 1600380.8, 1600381.6 and 160382.4 all filed Jan. 8, 2016, all of which are incorporated herein by reference in their entirety.

### INCORPORATION OF SEQUENCE LISTING

The instant application contains a Sequence Listing, named “ST26sequencelisting_KEMP0086USC3_N406716USC.XML” (78,790 bytes; created Jul. 22, 2025) which has been submitted electronically in ST.26 format and is hereby incorporated by reference in its entirety.

### FIELD OF THE INVENTION

The invention relates to an oncolytic immunotherapeutic agent and to the use of the oncolytic immunotherapeutic agent in treating cancer.

### BACKGROUND TO THE INVENTION

Viruses have a unique ability to enter cells at high efficiency. After entry into cells, viral genes are expressed and the virus replicates. This usually results in the death of the infected cell and the release of the antigenic components of the cell as the cell ruptures as it dies. As a result, virus mediated cell death tends to result in an immune response to these cellular components, including both those derived from the host cell and those encoded by or incorporated into the virus itself and enhanced due to the recognition by the host of so called damage associated molecular patterns (DAMPs) which aid in the activation of the immune response.

Viruses also engage with various mediators of the innate immune response as part of the host response to the recognition of a viral infection through e.g. toll-like receptors and cGAS/STING signalling and the recognition of pathogen associated molecular patterns (PAMPs) resulting in the activation of interferon responses and inflammation which are also immunogenic signals to the host. These immune responses may result in the immunogenic benefit to cancer patients such that immune responses to tumor antigens provide a systemic overall benefit resulting in the treatment of tumors which have not been infected with the virus, including micro-metastatic disease, and providing vaccination against relapse.

The combined direct (‘oncolytic’) effects of the virus, and immune responses against tumor antigens (including non-self ‘neo-antigens’, i.e. derived from the particular mutated genes in individual tumors) is termed ‘oncolytic immunotherapy’.

Viruses may also be used as delivery vehicles (‘vectors’) to express heterologous genes inserted into the viral genome in infected cells. These properties make viruses useful for a variety of biotechnology and medical applications. For example, viruses expressing heterologous therapeutic genes may be used for gene therapy. In the context of oncolytic immunotherapy, delivered genes may include those encoding specific tumor antigens, genes intended to induce immune responses or increase the immunogenicity of antigens released following virus replication and cell death, genes intended to shape the immune response which is generated, genes to increase the general immune activation status of the tumor, or genes to increase the direct oncolytic properties (i.e. cytotoxic effects) of the virus. Importantly, viruses have the ability to deliver encoded molecules which are intended to help to initiate, enhance or shape the systemic anti-tumor immune response directly and selectively to tumors, which may have benefits of e.g. reduced toxicity or of focusing beneficial effects on tumors (including those not infected by the virus) rather than off-target effects on normal (i.e. non-cancerous) tissues as compared to the systemic administration of these same molecules or systemic administration of other molecules targeting the same pathways.

It has been demonstrated that a number of viruses including, for example, herpes simplex virus (HSV) have utility in the oncolytic treatment of cancer. HSV for use in the oncolytic treatment of cancer must be disabled such that it is no longer pathogenic, but can still enter into and kill tumor cells. A number of disabling mutations to HSV, including disruption of the genes encoding ICP34.5, ICP6, and/or thymidine kinase, have been identified which do not prevent the virus from replicating in culture or in tumor tissue in vivo, but which prevent significant replication in normal tissue. HSVs in which only the ICP34.5 genes have been disrupted replicate in many tumor cell types in vitro, and replicate selectively in tumor tissue, but not in surrounding tissue, in mouse tumor models. Clinical trials of ICP34.5 deleted, or ICP34.5 and ICP6 deleted, HSV have also shown safety and selective replication in tumor tissue in humans.

As discussed above, an oncolytic virus, including HSV, may also be used to deliver a therapeutic gene in the treatment of cancer. An ICP34.5 deleted virus of this type additionally deleted for ICP47 and encoding a heterologous gene for GM-CSF has also been tested in clinical trials, including a phase 3 trial in melanoma in which safety and efficacy in man was shown. GM-CSF is a pro-inflammatory cytokine which has multiple functions including the stimulation of monocytes to exit the circulation and migrate into tissue where they proliferate and mature into macrophages and dendritic cells. GM-CSF is important for the proliferation and maturation of antigen presenting cells, the activity of which is needed for the activation of an anti-tumor immune response. The trial data demonstrated that tumor responses could be seen in injected tumors, and to a lesser extent in uninjected tumors. Responses tended to be highly durable (months-years), and a survival benefit appeared to be achieved in responding patients. Each of these indicated engagement of the immune system in the treatment of cancer in addition to the direct oncolytic effect. However, this and other data with oncolytic viruses generally showed that not all tumors respond to treatment and not all patients achieve a survival advantage. Thus, improvements to the art of oncolytic therapy are clearly needed.

Recently it has been shown that oncolytic immunotherapy can result in additive or synergistic therapeutic effects in conjunction with immune checkpoint blockade (i.e. inhibition or ‘antagonism’ of immune checkpoint pathways, also termed immune co-inhibitory pathways). Checkpoint (immune inhibitory pathway) blockade is intended to block host immune inhibitory mechanisms which usually serve to prevent the occurrence of auto-immunity. However, in cancer patients these mechanisms can also serve to inhibit the induction of or block the potentially beneficial effects of any immune responses induced to tumors.

Systemic blockade of these pathways by agents targeting CTLA-4, PD-1 or PD-L1 have shown efficacy in a number of tumor types, including melanoma and lung cancer. However, unsurprisingly, based on the mechanism of action, off target toxicity can occur due to the induction of auto-immunity. Even so, these agents are sufficiently tolerable to provide considerable clinical utility. Other immune co-inhibitory pathway and related targets for which agents (mainly antibodies) are in development include LAG-3, TIM-3, VISTA, CSF1R, IDO, CEACAM1, CD47. Optimal clinical activity of these agents, for example PD1, PDL1, LAG-3, TIM-3, VISTA, CSF1R, IDO, CD47, CEACAM1, may require systemic administration or presence in all tumors due to the mechanism of action, i.e. including targeting of the interface of immune effector cells with tumors or other immune inhibitory mechanisms in/of tumors. In some cases, more localised presence in e.g. just some tumors or in some lymph nodes may also be optimally effective, for example agents targeting CTLA-4.

An alternative approach to increasing the anti-tumor immune response in cancer patients is to target (activate) immune co-stimulatory pathways, i.e. in contrast to inhibiting immune co-inhibitory pathways. These pathways send activating signals into T cells and other immune cells, usually resulting from the interaction of the relevant ligands on antigen presenting cells (APCs) and the relevant receptors on the surface of T cells and other immune cells. These signals, depending on the ligand/receptor, can result in the increased activation of T cells and/or APCs and/or NK cells and/or B cells, including particular sub-types, increased differentiation and proliferation of T cells and/or APCs and/or NK cells and/or B cells, including particular subtypes, or suppression of the activity of immune inhibitory T cells such as regulatory T cells. Activation of these pathways would therefore be expected to result in enhanced anti-tumor immune responses, but it might also be expected that systemic activation of these pathways, i.e. activation of immune responses generally rather than anti-tumor immune responses specifically or selectively, would result in considerable off target toxicity in non-tumor tissue, the degree of such off target toxicity depending on the particular immune co-stimulatory pathway being targeted. Nevertheless agents (mainly agonistic antibodies, or less frequently the soluble ligand to the receptor in question) targeting immune co-stimulatory pathways, including agents targeting GITR, 4-1-BB, OX40, CD40 or ICOS, and intended for systemic use (i.e. intravenous delivery) are in or have been proposed for clinical development.

For many of these approaches targeting immune co-inhibitory or co-inhibitory pathways to be successful, pre-existing immune responses to tumors are needed, i.e. so that a pre-existing immune response can be potentiated or a block to an anti-tumor immune response can be relieved. The presence of an inflamed tumor micro-environment, which is indicative of such an ongoing response, is also needed. Pre-existing immune responses to tumor neo-antigens appear to be particularly important for the activity of immune co-inhibitory pathway blockade and related drugs. Only some patients may have an ongoing immune response to tumor antigens including neoantigens and/or an inflamed tumor microenvironment, both of which are required for the optimal activity of these drugs. Therefore, oncolytic agents which can induce immune responses to tumor antigens, including neoantigens, and/or which can induce an inflamed tumor microenvironment are attractive for use in combination with immune co-inhibitory pathway blockade and immune potentiating drugs. This likely explains the promising combined anti-tumor effects of oncolytic agents and immune co-inhibitory pathway blockade in mice and humans that have so far been observed.

The above discussion demonstrates that there is still much scope for improving oncolytic agents and cancer therapies utilising oncolytic agents, anti-tumor immune responses and drugs which target immune co-inhibitory or co-stimulatory pathways.

### SUMMARY OF THE INVENTION

The invention provides oncolytic viruses expressing GM-CSF and at least one molecule targeting an immune co-stimulatory pathway. GM-CSF aids in the induction of an inflammatory tumor micro-environment and stimulates the proliferation and maturation of antigen presenting cells, including dendritic cells, aiding the induction of an anti-tumor immune responses. These immune responses are amplified through activation of an immune co-stimulatory pathway or pathways using an immune co-stimulatory pathway activating molecule or molecules also delivered by the oncolytic virus.

The use of an oncolytic virus to deliver molecules targeting immune co-stimulatory pathways to tumors focuses the amplification of immune effects on anti-tumor immune responses, and reduces the amplification of immune responses to non-tumor antigens. Thus, immune cells in tumors and tumor draining lymph nodes are selectively engaged by the molecules activating immune co-stimulatory pathways rather than immune cells in general. This results in enhanced efficacy of immune co-stimulatory pathway activation and anti-tumor immune response amplification, and can also result in reduced off target toxicity. It is also important for focusing the effects of combined systemic immune co-inhibitory pathway blockade and immune co-stimulatory pathway activation on tumors, i.e. such that the amplified immune responses from which co-inhibitory blocks are released are antitumor immune responses rather than responses to non-tumor antigens.

The invention utilizes the fact that, when delivered by an oncolytic virus, the site of action of co-stimulatory pathway activation and of GM-CSF expression is in the tumor and/or tumor draining lymph node, but the results of such activation (an amplified systemic anti-tumor-immune response) are systemic. This targets tumors generally, and not only tumors to which the oncolytic virus has delivered the molecule or molecules targeting an immune co-stimulatory pathway or pathways and GM-CSF. Oncolytic viruses of the invention therefore provide improved treatment of cancer through the generation of improved tumor focused immune responses. The oncolytic virus of the invention also offers improved anti-tumor immune stimulating effects such that the immune-mediated effects on tumors which are not destroyed by oncolysis, including micro-metastatic disease, are enhanced, resulting in more effective destruction of these tumors, and more effective long term anti-tumor vaccination to prevent future relapse and improve overall survival.

Anti-tumor efficacy is improved when an oncolytic virus of the invention is used as a single agent and also when the virus is used in combination with other anti-cancer modalities, including chemotherapy, treatment with targeted agents, radiation and, in preferred embodiments, immune checkpoint blockade drugs (i.e. antagonists of an immune co-inhibitory pathway) and/or agonists of an immune co-stimulatory pathway.

Accordingly, the present invention provides an oncolytic virus comprising: (i) a GM-CSF-encoding gene; and (ii) an immune co-stimulatory pathway activating molecule or immune co-stimulatory pathway activating molecule-encoding gene. The virus may encode more than one immune co-stimulatory pathway activating molecule/gene.

The immune co-stimulatory pathway activating molecule is preferably GITRL, 4-1-BBL, OX40L, ICOSL or CD40L or a modified version of any thereof or a protein capable of blocking signaling through CTLA-4, for example an antibody which binds CTLA-4. Examples of modified versions include agonists of a co-stimulatory pathway that are secreted rather than being membrane bound, and/or agonists modified such that multimers of the protein are formed.

The virus may be a modified clinical isolate, such as a modified clinical isolate of a virus, wherein the clinical isolate kills two or more tumor cell lines more rapidly and/or at a lower dose in vitro than one or more reference clinical isolates of the same species of virus.

The virus is preferably a herpes simplex virus (HSV), such as HSV1. The HSV typically does not express functional ICP34.5 and/or functional ICP47 and/or expresses the US11 gene as an immediate early gene.

The invention also provides:
a pharmaceutical composition comprising a virus of the invention and a pharmaceutically acceptable carrier or diluent;
the virus of the invention for use in a method of treating the human or animal body by therapy;
the virus of the invention for use in a method of treating cancer, wherein the method optionally comprises administering a further anti-cancer agent;
a product of manufacture comprising a virus of the invention in a sterile vial, ampoule or syringe;
a method of treating cancer, which comprises administering a therapeutically effective amount of a virus or a pharmaceutical composition of the invention to a patient in need thereof, wherein the method optionally comprises administering a further anti-cancer agent which is optionally an antagonist of an immune co-inhibitory pathway, or an agonist of an immune co-stimulatory pathway;
use of a virus of the invention in the manufacture of a medicament for use in a method of treating cancer, wherein the method optionally comprises administering a further anti-cancer agent which is optionally an antagonist of an immune co-inhibitory pathway, or an agonist of an immune co-stimulatory pathway;
a method of treating cancer, which comprises administering a therapeutically effective amount of an oncolytic virus, an inhibitor of the indoleamine 2,3-dioxygenase (IDO) pathway and a further antagonist of an immune co-inhibitory pathway, or an agonist of an immune co-stimulatory pathway to a patient in need thereof.

### BRIEF DESCRIPTION OF THE FIGURES

FIG. 1 depicts the structure of an exemplary virus of the invention that comprises a gene encoding GM-CSF and a gene encoding CD40L.

FIG. 2 shows the differential abilities of the eight top ranking HSV1 clinical isolate strains as assessed by crystal violet staining 24 hours or 48 hours after infection with a MOI of 0.1, 0.01 or 0.001 as indicated in the Figure to kill Fadu, SK-mel-28, A549, HT1080, MIA-PA-CA-2, HT29 and MDA-MB-231 human tumor cell lines. The virus strains ranked first and second on each cell line are indicated. The virus RH018A was ranked first on each of the Fadu, HT1080, MIA-PA-CA-2 and HT29 cell lines and second on each of the SK-mel-28, A549 and MDA-MB-231 cell lines. RH004A was ranked joint first with RH018A and RH015A on the HT29 cell line, first on the SK-mel-28 and AS49 cell lines and second on the Fadu cell line. RH023A was ranked first on the MDA-MB-231 cell line and second on the HT1080 cell line. RH031A was ranked second on each of the MIA-PA-CA-2 and HT29 cell lines. RH040A was ranked joint second on the HT29 cell line.

FIG. 3 shows a comparison between strain RH018A, the strain ranked first of all the strains tested, with an ‘average’ strain from the screen (i.e. strain RH065A). Approximately 10 fold less of strain RH018A was needed to kill an equal proportion of cells than was needed of strain RH065A as shown by crystal violet staining 24 or 48 hours post infection with MOIs of 0.1, 0.01 and 0.001 in SK-mel-28, HT1080, MDA-MB-231, Fadu, MIA-PA-CA-2 and A549 cell lines.

FIGS. 4A-4F and 5A-5F depict structures of HSV1 viruses modified by the deletion of ICP34.5 and ICP47 such that the US11 gene is under control of the ICP457 immediate early promoter and containing heterologous genes in the ICP34.5 locus. The viruses were constructed using the RH018A strain unless otherwise stated in the Figure.

FIG. 6 shows the results of an ELISA to detect expression of human or mouse GM-CSF in supernatants from BHK cells infected with virus 16 (mGM-CSF and GALVR-), virus 17 (hGM-CSF and GALVR-) and virus 19 (mGM-CSF).

FIGS. 7A-7B is a comparison between the cell-killing abilities of strain RH018A in which ICP34.5 is deleted and which expresses GALVR- and GFP (virus 10) with a virus that expresses only GFP (virus 12) as determined by crystal violet staining in three cell lines at low magnification.

FIGS. 8A-8B is a comparison between the cell-killing abilities of strain RH018A in which ICP34.5 and ICP47 are deleted and which expresses GALVR- and GM-CSF (virus 17) with a prior art strain with the same modifications as determined by crystal violet staining in four cell lines.

FIG. 9 shows the effectiveness of Virus 16 (ICP34.5 and ICP47 deleted expressing GALVR- and mGM-CSF) in treating mice harbouring A20 lymphoma tumors in both flanks. Tumors on the right flanks were injected with the virus or vehicle and the effects on tumor size was observed for 30 days. The virus was effective against both injected tumors and non-injected tumors.

FIG. 10 demonstrates the effects of Virus 15 (ICP34.5 and ICP47 deleted expressing GALVR- and GFP) and Virus 24 (ICP34.5 and ICP47 deleted expressing GFP) on rat 9L cells in vitro as assessed by crystal violet staining. The virus expressing GALV (Virus 15) showed enhanced killing of rat 9L cells in vitro as compared to a virus which does not express GALV (Virus 24).

FIGS. 11A-11C shows the antitumor effects of Virus 16 in Balb/c mice harboring mouse CT26 tumors in the left and right flanks. Groups of 10 mice were then treated with: Vehicle (3 injections into right flank tumors every other day); 5×10exp6 pfu of Virus 16 (mRP1) injected in the right flank tumor every other day; anti-mouse PD1 alone (10 mg/kg i.p. every three days, BioXCell clone RMP1-14); anti-mouse CTLA-4 (3 mg/kg i.p every three days, BioXCell clone 9D9); anti-mouse PD1 together with Virus 16; anti-mouse CTLA4 together with Virus 16; 1-methyl trypotophan (I-MT; IDO inhibitor (5 mg/ml in drinking water)); anti-mouse PD1 together with 1-methyl trypotophan; or anti-mouse PD1 together with 1-methyl trypotophan and Virus 16. Effects on tumor size were observed for a further 30 days. Greater tumor reduction was seen in animals treated with combinations of virus and checkpoint bockade than with the single treatment groups. FIG. 11A shows that using Virus 16 and anti-PD1 in combination has a better anti-tumor effect than using either anti-PD1 or the virus alone. FIG. 11B shows that the anti-tumor effect of Virus 16 in combination with anti-CTLA-4 was better than the anti-tumor effect of either Virus 16 or anti-CTLA-4 alone. FIG. 11C shows that enhanced tumor reduction was observed using Virus 16 together with both anti-PD1 and IDO inhibition as compared to anti-PD1 and 1-MT inhibition in the absence of the virus.

FIGS. 12A-12D shows the enhanced anti-tumor activity of Virus 16 in combination with immune checkpoint blockade in mouse A20 tumors in both flanks of Balb/c mice as compared to either virus alone or checkpoint blockade alone (anti-PD1).

FIG. 13 shows the structure of ICP34.5 and ICP47 deleted viruses expressing GALVR-, GM-CSF and codon optimized anti-mouse or anti-human CTLA-4 antibody constructs (secreted scFv molecules linked to human or mouse IgG1 Fc regions). The scFvs contain the linked ([G₄S]₃) light and heavy variable chains from antibody 9D9 (U.S. 2011044953: mouse version) and from ipilimumab (U.S. 20150283234; human version). The resulting structure of the CTLA-4 inhibitor is also shown.

FIG. 14 shows anti-tumor effects of Virus 16 and Virus 19 in a human xenograft model (A549). There were three injections of Virus 16, Virus 19 or of vehicle over one week at three different dose levels (N=10/group). The doses of the viruses used is indicated. The anti-tumor effects of Virus 16 which expresses GALV were better than those of Virus 19 which does not express GALV.

FIG. 15 demonstrates the effects of viruses of the invention expressing GALVR- on 9L cells in the flanks of Fischer 344 rats. The following treatments were administered to groups of rats (ten per group), into one flank of each rat only three times per week for three weeks: 50 μl of vehicle; 50 μl of 107 pfu/ml of Virus 19 (expresses mGM-CSF but not GALV R-); or 50μl of 10⁷ pfu/ml of Virus 16 (expresses both mouse GM-CSF and GALV-R-). Effects on tumor growth were then observed for a further 30 days. Superior tumor control and shrinkage was observed with the virus expressing GM-CSF and GALV-R-as compared to the virus expressing GM-CSF alone.

FIG. 16 shows the anti-tumor effects of viruses expressing anti-mCTLA-4 (virus 27), mCD40L (virus 32), mOX4OL (virus 35), m4-2BBL (virus 33), each also with mGM-CSF and GALV-R-compared to virus 16 (expresses GALV and mGM-CSF).

### BRIEF DESCRIPTION OF THE SEQUENCE LISTING

SEQ ID NO: 1 is the nucleotide sequence of mouse GM-CSF.
SEQ ID NO: 2 is the nucleotide sequence of a codon optimized version of mouse GM-CSF.
SEQ ID NO: 3 is the nucleotide sequence of human GM-CSF.
SEQ ID NO: 4 is the nucleotide sequence of a codon optimized version of human GM-CSF.
SEQ ID NO: 5 is the amino acid sequence of mouse GM-CSF.
SEQ ID NO: 6 is the amino acid sequence of human GM-CSF.
SEQ ID NO: 7 is the nucleotide sequence of GALV-R-.
SEQ ID NO: 8 is the nucleotide sequence of a codon optimized version of GALV-R- (the first three nucleotides are optional)
SEQ ID NO: 9 is the amino acid sequence of GALV-R-.
SEQ ID NO: 10 is the nucleotide sequence of a codon optimized version of a human membrane bound version of CD40L.
SEQ ID NO: 11 is the amino acid sequence of a human membrane bound version of CD40L.
SEQ ID NO: 12 is the nucleotide sequence of a codon optimized version of a multimeric secreted version of human CD40L.
SEQ ID NO: 13 is the amino acid sequence of a multimeric secreted version of human CD40L.
SEQ ID NO: 14 is the nucleotide sequence of a codon optimized version of a multimeric secreted version of mouse CD40L.
SEQ ID NO: 15 is the amino acid sequence of a multimeric secreted version of mouse CD40L.
SEQ ID NO: 16 is a codon optimized version of the nucleotide sequence of wild-type human CD40L.
SEQ ID NO: 17 is the amino acid sequence of wild-type human CD40L.
SEQ ID NO: 18 is a codon optimized version of the nucleotide sequence of wild-type mouse CD40L.
SEQ ID NO: 19 is the amino acid sequence of wild-type mouse CD40L.
SEQ ID NO: 20 is the nucleotide sequence of a codon optimized version of murine 4-1BBL.
SEQ ID NO: 21 is the nucleotide sequence of a codon optimized version of human 4-1BBL.
SEQ ID NO: 22 is the nucleotide sequence of a codon optimized version of secreted mouse 4-1BBL.
SEQ ID NO: 23 is the nucleotide sequence of a codon optimized version of human secreted 4-1BBL.
SEQ ID NO: 24 is the nucleotide sequence of a codon optimized version of murine GITRL.
SEQ ID NO: 25 is the nucleotide sequence of a codon optimized version of human GITRL.
SEQ ID NO: 26 is the nucleotide sequence of a codon optimized version of secreted murine GITRL.
SEQ ID NO: 27 is the nucleotide sequence of a codon optimized version of secreted human GITRL.
SEQ ID NO: 28 is the nucleotide sequence of a codon optimized version of murine OX40L.
SEQ ID NO: 29 is the nucleotide sequence of a codon optimized version of human OX40L.
SEQ ID NO: 30 is the nucleotide sequence of a codon optimized version of secreted murine OX40L.
SEQ ID NO: 31 is the nucleotide sequence of a codon optimized version of secreted human OX40L.
SEQ ID NO: 32 is the nucleotide sequence of a codon optimized version of murine ICOSL.
SEQ ID NO: 33 is the nucleotide sequence of a codon optimized version of human ICOSL.
SEQ ID NO: 34 is the nucleotide sequence of a murine scFv CTLA-4 antibody. The first six and last eight nucleotides are restriction sites added for cloning purposes.
SEQ ID NO: 35 is the nucleotide sequence of a murine scFv CTLA-4 antibody. The first six and last eight nucleotides are restriction sites added for cloning purposes.
SEQ ID NO: 36 is the nucleotide sequence of the CMV promoter.
SEQ ID NO: 37 is the nucleotide sequence of the RSV promoter.
SEQ ID NO: 38 is the nucleotide sequence of BGH polyA.
SEQ ID NO: 39 is the nucleotide sequence of SV40 late polyA.
SEQ ID NO: 40 is the nucleotide sequence of the SV40 enhancer promoter.
SEQ ID NO: 41 is the nucleotide sequence of rabbit beta-globulin (RBG) polyA.
SEQ ID NO: 42 is the nucleotide sequence of GFP.
SEQ ID NO: 43 is the nucleotide sequence of the MoMuLV LTR promoter.
SEQ ID NO: 44 is the nucleotide sequence of the EF1a promoter.
SEQ ID NO: 45 is the nucleotide sequence of HGH polyA.

### DETAILED DESCRIPTION OF THE INVENTION

### Oncolytic Virus

The virus of the invention is oncolytic. An oncolytic virus is a virus that infects and replicates in tumor cells, such that the tumor cells are killed. Therefore, the virus of the invention is replication competent. Preferably, the virus is selectively replication competent in tumor tissue. A virus is selectively replication competent in tumor tissue if it replicates more effectively in tumor tissue than in non-tumor tissue. The ability of a virus to replicate in different tissue types can be determined using standard techniques in the art.

The virus of the invention may be any virus which has these properties, including a herpes virus, pox virus, adenovirus, retrovirus, rhabdovirus, paramyxovirus or reovirus, or any species or strain within these larger groups. Viruses of the invention may be wild type (i.e. unaltered from the parental virus species), or with gene disruptions or gene additions. Which of these is the case will depend on the virus species to be used. Preferably the virus is a species of herpes virus, more preferably a strain of HSV, including strains of HSV1 and HSV2, and is most preferably a strain of HSV1. In particularly preferred embodiments the virus of the invention is based on a clinical isolate of the virus species to be used. The clinical isolate may have been selected on the basis of it having particular advantageous properties for the treatment of cancer.

The clinical isolate may have surprisingly good anti-tumor effects compared to other strains of the same virus isolated from other patients, wherein a patient is an individual harbouring the virus species to be tested. The virus strains used for comparison to identify viruses of the invention may be isolated from a patient or an otherwise healthy (i.e. other than harboring the virus species to be tested) volunteer, preferably an otherwise healthy volunteer. HSV1 strains used to identify a virus of the invention are typically isolated from cold sores of individuals harboring HSV1, typically by taking a swab using e.g. Virocult (Sigma) brand swab/container containing transport media followed by transport to the facility to be used for further testing.

After isolation of viruses to be compared from individuals, stocks of the viruses are typically prepared, for example by growing the isolated viruses on BHK or vero cells. Preferably, this is done following no more than 3 cycles of freeze thaw between taking the sample and it being grown on, for example, BHK or vero cells to prepare the virus stock for further use. More preferably the virus sample has undergone 2 or less than 2 cycles of freeze thaw prior to preparation of the stock for further use, more preferably one cycle of freeze thaw, most preferably no cycles of freeze thaw. Lysates from the cell lines infected with the viruses prepared in this way after isolation are compared, typically by testing for the ability of the virus to kill tumor cell lines in vitro. Alternatively, the viral stocks may be stored under suitable conditions, for example by freezing, prior to testing. Viruses of the invention have surprisingly good anti-tumor effects compared to other strains of the same virus isolated from other individuals, preferably when compared to those isolated from >5 individuals, more preferably >10 other individuals, most preferably >20 other individuals.

The stocks of the clinical isolates identified for modification to produce viruses of the invention (i.e. having surprisingly good properties for the killing of tumor cells as compared to other viral strains to which they were compared) may be stored under suitable conditions, before or after modification, and used to generate further stocks as appropriate.

A clinical isolate is a strain of a virus species which has been isolated from its natural host. The clinical isolate has preferably been isolated for the purposes of testing and comparing the clinical isolate with other clinical isolates of that virus species for a desired property, in the case of viruses of the invention that being the ability to kill human tumor cells. Clinical isolates which may be used for comparison also include those from clinical samples present in clinical repositories, i.e. previously collected for clinical diagnostic or other purposes. In either case the clinical isolates used for comparison and identification of viruses of the invention will preferably have undergone minimal culture in vitro prior to being tested for the desired property, preferably having only undergone sufficient culture to enable generation of sufficient stocks for comparative testing purposes. As such, the viruses used for comparison to identify viruses of the invention may also include deposited strains, wherein the deposited strain has been isolated from a patient, preferably an HSV1 strain isolated from the cold sore of a patient.

The virus may be a modified clinical isolate, wherein the clinical isolate kills two or more tumor cell lines more rapidly and/or at a lower dose in vitro than one or more reference clinical isolate of the same species of virus. Typically, the clinical isolate will kill two or more tumor cell lines within 72 hours, preferably within 48 hours, more preferably within 24 hours, of infection at multiplicities of infection (MOI) of less than or equal to 0.1, preferably less than or equal to an MOI of 0.01, more preferably less than or equal to an MOI of 0.001. Preferably the clinical isolate will kill a broad range of tumor cell lines, such as 2, 3, 4, 5, 6, 7, 8, 9, 10 or, for example, all of the following human tumor cell lines: U87MG (glioma), HT29 (colorectal), LNCaP (prostate), MDA-MB-231 (breast), SK-MEL-28 (melanoma), Fadu (squamous cell carcinoma), MCF7 (breast), A549 (lung), MIAPACA-2 (pancreas), CAPAN-1 (pancreas), HT1080 (fibrosarcoma).

Thus, the virus of the invention may be capable of killing cells from two or more, such as 3, 4, 5, 6, 7 or more, different types of tumor such as two or more, such as 3, 4, 5, 6, 7 or more, solid tumors, including but not limited to colorectal tumor cells, prostate tumor cells, breast tumor cells, ovarian tumor cells, melanoma cells, squamous cell carcinoma cells, lung tumor cells, pancreatic tumor cells, sarcoma cells and/or fibrosarcoma cells.

Tumor cell line killing can be determined by any suitable method. Typically, a sample is first isolated from a patient, preferably, in the case of HSV1, from a cold sore, is used to infect BHK cells, or another suitable cell line such as vero cells. Positive samples are typically identified by the presence of a cytopathic effect (CPE) 24-72 hours post infection, such as 48 hours post infection, and confirmed to be the target viral species by, for example, immunohistochemistry or PCR. Viral stocks are then generated from the positive samples. A sample from the viral stock is typically tested and compared to other samples generated in the same way using swabs from different patients. Testing may be carried out by determining the level of CPE achieved at a range of multiplicity of infection (MOI) and at various times post infection.

For example, cell lines at 80% confluency may be infected with the viral sample at MOI of 1, 0.1, 0.01 and 0.001 and duplicate plates incubated for 24 and 48 hours at 37° C., 5% CO₂ prior to determination of the extent of viral cell killing. This may be determined by, for example, fixing the cells with glutaraldehyde and staining with crystal violet using standard methods. The level of cell lysis may then be assessed by standard methods such as gross observation, microscopy (cell counts) and photography. The method may be repeated with the cells being incubated for shorter time periods, such as 8, 12 or 16 hours, or longer time periods, such as 72 hours, before cell killing is determined, or at additional MOIs such as 0.0001 or less.

Growth curve experiments may also be conducted to assess the abilities of different clinical isolates to replicate in tumor cell lines in vitro. For example, cell lines at 80% confluency may be infected with the viral sample at MOI of 1, 0.1, 0.01 and 0.001 are incubated at 37° C., 5% CO₂ and the cells lysed, typically by freeze/thawing, at 0, 8, 16, 24 and 48 hours post infection prior to determination of the extent of viral cell killing. This may be determined by, for example, assessing viral titres by a standard plaque assay.

A clinical isolate of the invention can kill infected tumor cell lines more rapidly and/or at a lower MOI than the other clinical isolates to which it is compared, preferably 2, 3, 4, 5 or 10 or more, other clinical isolates of the same virus species. The clinical isolate of the invention typically kills a 10%, 25% or 50% greater proportion of the tumor cells present at a particular MOI and time point than at least one, preferably 2, 3, 4, 5 or 10 or more, other clinical isolates of the same virus type at the same MOI and time point to which it was compared. The clinical isolate of the invention typically kills the same or a greater proportion of tumor cells at a MOI that is half or less than half that of the MOI at which one or more, preferably 2, 3, 4, 5,10 or 15 or more, other clinical isolates of the same virus species used for the comparison at the same time point, typically at 12, 24 and/or 48 hours, kills the same proportion of tumor cells. Preferably, a clinical isolate of the invention typically kills the same or a greater proportion of tumor cells at a MOI that is 5 or 10 times lower than the MOI at which one or more, preferably 2, 3, 4, 5, 10 or 15 or more, other clinical isolates of the same virus used for the comparison at the same time point, typically at 12, 24 and/or 48 hours kills the same proportion of tumor cells. The improved tumor cell killing abilities of a virus of the invention are typically achieved compared to at least 50%, 75% or 90% of the other clinical isolates of the same viral species used for the comparison. The virus is preferably compared to at least 4 other virus strains, such as, for example, 7, 9, 19, 39 or 49 other virus strains of the same species.

The isolated strains may be tested in batches, for example of 4-8 viral strains at a time, on, for example, 4-8 of the tumor cell lines at a time. For each batch of experiments, the degree of killing achieved is ranked on each cell line for the best (i.e. least surviving cells at each time point/MOI) to the worst (i.e. most surviving cells for each time point/MOI) for the viruses being compared in that experiment. The virus strains from each experiment which perform the best across the range of tumor cell line tested (i.e. that consistently ranked as one of the best at killing the cell lines) may then be compared head to head in further experiments using other clinical isolates and/ore other tumor cell lines to identify the best virus strains in the total of, for example, >20 virus strains sampled. Those ranked as the best overall are the viruses of the invention.

In a preferred embodiment, the virus of the invention is a strain selected from:
strain RH018A having the provisional accession number ECCAC 16121904;
strain RH004A having the provisional accession number ECCAC 16121902;
strain RH031A having the provisional accession number ECCAC 16121907;
strain RH040B having the provisional accession number ECCAC 16121908;
strain RH015A having the provisional accession number ECCAC 16121903;
strain RH021A having the provisional accession number ECCAC 16121905;
strain RH023A having the provisional accession number ECCAC 16121906; and
strain RH047A having the provisional accession number ECCAC 16121909.

More preferably, the virus of the invention is a strain selected from:
strain RH018A having the provisional accession number ECCAC 16121904;
strain RH004A having the provisional accession number ECCAC 16121902;
strain RH031A having the provisional accession number ECCAC 16121907;
strain RH040B having the provisional accession number ECCAC 16121908; and
strain RH015A having the provisional accession number ECCAC 16121903;

Most preferably, the virus of the invention is strain RH018A having the accession number EACC 16121904.

An HSV of the invention is capable of replicating selectively in tumors, such as human tumors. Typically, the HSV replicates efficiently in target tumors but does not replicate efficiently in non-tumor tissue. This HSV may comprise one or more mutations in one or more viral genes that inhibit replication in normal tissue but still allow replication in tumors. The mutation may, for example, be a mutation that prevents the expression of functional ICP34.5, ICP6 and/or thymidine kinase by the HSV.

In one preferred embodiment, the ICP34.5-encoding genes are mutated to confer selective oncolytic activity on the HSV. Mutations of the ICP34.5-encoding genes that prevent the expression of functional ICP34.5 are described in Chou et al. (1990) Science 250:1262-1266, Maclean et al. (1991) J. Gen. Virol. 72:631-639 and Liu et al. (2003) Gene Therapy 10:292-303, which are incorporated herein by reference. The ICP6-encoding gene and/or thymidine kinase-encoding gene may also be inactivated, as may other genes provided that such inactivation does not prevent the virus infecting or replicating in tumors.

The HSV may contain a further mutation or mutations which enhance replication of the HSV in tumors. The resulting enhancement of viral replication in tumors not only results in improved direct ‘oncolytic’ tumor cell killing by the virus, but also enhances the level of heterologous (i.e. a gene inserted into the virus, in the case of viruses of the invention genes encoding GM-CSF and an immune co-stimulatory pathway activating molecule(s)) gene expression and increases the amount of tumor antigen released as tumor cells die, both of which may also improve the immunogenic properties of the therapy for the treatment of cancer. For example, in a preferred embodiment of the invention, deletion of the ICP47-encoding gene in a manner that places the US11 gene under the control of the immediate early promoter that normally controls expression of the ICP47 encoding gene leads to enhanced replication in tumors (see Liu et al., 2003, which is incorporated herein by reference).

Other mutations that place the US11 coding sequence, which is an HSV late gene, under the control of a promoter that is not dependent on viral replication may also be introduced into a virus of the invention. Such mutations allow expression of US11 before HSV replication occurs and enhance viral replication in tumors. In particular, such mutations enhance replication of an HSV lacking functional ICP34.5-encoding genes.

Accordingly, in one embodiment the HSV of the invention comprises a US11 gene operably linked to a promoter, wherein the activity of the promoter is not dependent on viral replication. The promoter may be an immediate early (IE) promoter or a non-HSV promoter which is active in mammalian, preferably human, tumor cells. The promoter may, for example, be a eukaryotic promoter, such as a promoter derived from the genome of a mammal, preferably a human. The promoter may be a ubiquitous promoter (such as a promoter of β-actin or tubulin) or a cell-specific promoter, such as tumor-specific promoter. The promoter may be a viral promoter, such as the Moloney murine leukaemia virus long terminal repeat (MMLV LTR) promoter or the human or mouse cytomegalovirus (CMV) IE promoter. HSV immediate early (IE) promoters are well known in the art. The HSV IE promoter may be the promoter driving expression of ICP0, ICP4, ICP22, ICP27 or ICP47.

The genes referred to above the functional inactivation of which provides the property of tumor selectivity to the virus may be rendered functionally inactive by any suitable method, for example by deletion or substitution of all or part of the gene and/or control sequence of the gene or by insertion of one or more nucleic acids into or in place of the gene and/or the control sequence of the gene. For example, homologous recombination methods, which are standard in the art, may be used to generate the virus of the invention. Alternatively bacterial artificial chromosome (BAC)-based approaches may be used.

As used herein, the term “gene” is intended to mean the nucleotide sequence encoding a protein, i.e. the coding sequence of the gene. The various genes referred to above may be rendered non-functional by mutating the gene itself or the control sequences flanking the gene, for example the promoter sequence. Deletions may remove one or more portions of the gene, the entire gene or the entire gene and all or some of the control sequences. For example, deletion of only one nucleotide within the gene may be made, resulting in a frame shift. However, a larger deletion may be made, for example at least about 25%, more preferably at least about 50% of the total coding and/or non-coding sequence. In one preferred embodiment, the gene being rendered functionally inactive is deleted. For example, the entire gene and optionally some of the flanking sequences may be removed from the virus. Where two or more copies of the gene are present in the viral genome both copies of the gene are rendered functionally inactive.

A gene may be inactivated by substituting other sequences, for example by substituting all or part of the endogenous gene with a heterologous gene and optionally a promoter sequence. Where no promoter sequence is substituted, the heterologous gene may be inserted such that it is controlled by the promoter of the gene being rendered non-functional. In an HSV of the invention it is preferred that the ICP34.5 encoding-genes are rendered non-functional by the insertion of a heterologous gene or genes and a promoter sequence or sequences operably linked thereto, and optionally other regulatory elements such as polyadenylation sequences, into each the ICP34.5-encoding gene loci.

A virus of the invention is used to express GM-CSF and an immune co-stimulatory pathway activating molecule in tumors. This is typically achieved by inserting a heterologous gene encoding GM-CSF and a heterologous gene encoding the immune co-stimulatory pathway activating molecule in the genome of a selectively replication competent virus wherein each gene is under the control of a promoter sequence. As replication of such a virus will occur selectively in tumor tissue, expression of the GM-CSF and the immune co-stimulatory activating protein by the virus is also enhanced in tumor tissue as compared to non-tumor tissue in the body. Enhanced expression occurs where expression is greater in tumors as compared to other tissues of the body. Proteins expressed by the oncolytic virus would also be expected to be present in oncolytic virus-infected tumor draining lymph nodes, including due to trafficking of expressed protein and of virus in and on antigen presenting cells from the tumor. Accordingly, the invention provides benefits of expression of both GM-CSF and an immune co-stimulatory pathway activating molecule selectively in tumors and tumor draining lymph nodes combined with the anti-tumor effect provided by oncolytic virus replication.

The virus of the invention comprises GM-CSF. The sequence of the gene encoding GM-CSF may be codon optimized so as to increase expression levels of the respective proteins in target cells as compared to if the unaltered sequence is used.

The virus of the invention comprises one or more immune co-stimulatory pathway activating molecules and/or one or more genes encoding an immune co-stimulatory pathway activating molecule. Immune co-stimulatory pathway activating molecules include proteins and nucleic acid molecules (e.g. aptamer sequences). Examples of immune co-stimulatory pathway activating molecules include CD40 ligand, GITR ligand, 4-1-BB ligand, OX40 ligand, ICOS ligand, flt3 ligand, TL1A, CD30 ligand, CD70 and single chain antibodies targeting the respective receptors for these molecules (CD40, GITR, 4-1-BB, OX40, ICOS, flt3, DR3, CD30, CD27). The CD40L, GITRL, 4-1-BBL, OX40L, ICOSL, ft3L, TL1A, CD30L or CD70L may be a modified version of any thereof, such as a soluble version.

Activators of immune co-stimulatory pathways include mutant or wild type, soluble, secreted and/or membrane bound ligands, and agonistic antibodies including single chain antibodies. Viruses of the invention preferably encode one or more of CD40L, ICOSL, 4-1-BBL, GITRL or OX40L.

The inhibitor of a co-inhibitory pathway may be a CTLA-4 inhibitor. The CTLA-4 inhibitor is typically a molecule such as a peptide or protein that binds to CTLA-4 and reduces or blocks signaling through CTLA-4, such as by reducing activation by B7. By reducing CTLA-4 signalling, the inhibitor reduces or removes the block of immune stimulatory pathways by CTLA-4.

The CTLA-4 inhibitor is preferably an antibody or an antigen binding fragment thereof. The term “antibody” as referred to herein includes whole antibodies and any antigen binding fragment (i.e., “antigen-binding portion”) or single chains thereof. An antibody refers to a glycoprotein comprising at least two heavy (H) chains and two light (kappa)(L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g., effector cells) and the first component (Clq) of the classical complement system.

The antibody is typically a monoclonal antibody. The antibody may be a chimeric antibody. The antibody is preferably a humanised antibody and is more preferably a human antibody.

The term “antigen-binding fragment” of an antibody refers to one or more fragments of an antibody that retain the ability to specifically bind to CTLA-4. The antigen-binding fragment also retains the ability to inhibit CTLA-4 and hence to reduce or remove the CTLA-4 blockade of a stimulatory immune response. Examples of suitable fragments include a Fab fragment, a F(ab′)₂ fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are also intended to be encompassed within the term “antigen-binding portion” of an antibody. In a preferred embodiment, the antibody is an scFv. Examples of suitable scFv molecules are disclosed in, for example, WO2007/123737 and WO2014/066532, which are incorporated herein by reference. The scFv may be encoded by the nucleotide sequence shown in SEQ ID NO: 34 the nucleotide sequence shown in SEQ ID NO: 35.

Viruses of the invention may encode one or more immune co-stimulatory pathway activating molecules, preferably 1, 2, 3 or 4 immune co-stimulatory pathway activating molecules, more preferably 1 or 2 immune co-stimulatory pathway activating molecules.

For example, the virus may comprise genes encoding:
CD40L and one or more of ICOSL, 4-1-BBL, GITRL, OX40L and a CTLA-4 inhibitor;
ICOSL and one or more of CD40L, 4-1-BBL, GITRL, OX40L and a CTLA-4 inhibitor;
4-1-BBL and one or more of CD40L, ICOSL, GITRL, OX40L and a CTLA-4 inhibitor;
GITRL and one or more of CD40L, ICOSL, 4-1-BBL, OX40L and a CTLA-4 inhibitor;
OX40L and one or more of CD40L, ICOSL, 4-1-BBL, GITRL and a CTLA-4 inhibitor;
a CTLA-4 inhibitor and one or more of CD40L, ICOSL, 4-1-BBL, GITRL and OX40L.

The sequence of the gene encoding the immune co-stimulatory activating molecule may be codon optimized so as to increase expression levels of the respective protein(s) in target cells as compared to if the unaltered sequence is used.

The virus of the invention may comprise one or more further heterologous genes in addition to GM-CSF and an immune co-stimulatory pathway activating molecule, including, in a preferred embodiment, a fusogenic protein such as GALVR-.

The fusogenic protein may be any heterologous protein capable of promoting fusion of a cell infected with the virus of the invention to another cell. A fusogenic protein, preferably a wild type or modified viral glycoprotein (i.e. modified to increase its fusogenic properties), is a protein which is capable in inducing the cell to cell fusion (syncitia formation) of cells in which it is expressed. Examples of fusogenic glycoproteins include VSV-G, syncitin-1 (from human endogenous retrovirus-W (HERV-W)) or syncitin-2 (from HERVFRDE1), paramyxovirus SV5-F, measles virus-H, measles virus-F, RSV-F, the glycoprotein from a retrovirus or lentivirus, such as gibbon ape leukemia virus (GALV), murine leukemia virus (MLV), Mason-Pfizer monkey virus (MPMV) and equine infectious anemia virus (EIAV) with the R transmembrane peptide removed (R-versions). In a preferred embodiment the fusogenic protein is from GALV and has the R-peptide removed (GALV-R-).

The virus of the invention may optionally comprise multiple copies of the fusogenic protein-encoding gene, preferably 1 or 2 copies. The virus may comprise two or more different fusogenic proteins, including any of the fusogenic proteins listed above.

The fusogenic protein or proteins optionally expressed by a virus of the invention may be identical to a naturally occurring protein, or may be a modified protein.

The fusogenic protein-encoding gene (fusogenic gene) may have a naturally occurring nucleic acid sequence or a modified sequence. The sequence of the fusogenic gene may, for example, be modified to increase the fusogenic properties of the encoded protein, or to provide codon optimisation and therefore increase the efficiency of expression of the encoded protein.

The invention also provides a virus, such as a pox virus or a HSV, preferably HSV1, which expresses at least three heterologous genes, wherein each of the three heterologous genes is driven by a different promoter selected from the CMV promoter, the RSV promoter, the EF1a promoter, the SV40 promoter and a retroviral LTR promoter. The virus may, for example, express four heterologous genes, wherein each of the four heterologous genes is driven by a different promoter selected from the CMV promoter, the RSV promoter, the EF1a promoter, the SV40 promoter and a retroviral LTR promoter. The retroviral LTR is preferably from MMLV (SEQ ID NO:43), also known as MoMuLV. The heterologous genes may be terminated by poly adenylation sequences. The poly adenylation sequences may be the same or different. Preferably each heterologous gene is terminated by a different poly adenylation sequence, which is preferably selected from the BGH, SV40, HGH and RBG poly adenylation sequences.

The invention also provides a virus, such as a pox virus or a HSV, preferably HSV1, which expresses at least three heterologous genes, wherein each of the three heterologous genes is terminated by a different poly adenylation sequence selected from the BGH, SV40, HGH and RBG poly adenylation sequences. The virus may, for example, express four heterologous genes terminated by each of the BGH, SV40, HGH and RBG poly adenylation sequences, respectively.

### Production of Virus

Viruses of the invention are constructed using methods well known in the art. For example plasmids (for smaller viruses and single and multiple genome component RNA viruses) or BACs (for larger DNA viruses including herpes viruses) encoding the viral genome to be packaged, including the genes encoding the fusogenic and immune stimulating molecules under appropriate regulatory control, can be constructed by standard molecular biology techniques and transfected into permissive cells from which recombinant viruses can be recovered.

Alternatively, in a preferred embodiment plasmids containing DNA regions flanking the intended site of insertion can be constructed, and then co-transfected into permissive cells with viral genomic DNA such that homologous recombination between the target insertion site flanking regions in the plasmid and the same regions in the parental virus occur. Recombinant viruses can then be selected and purified through the loss or addition of a function inserted or deleted by the plasmid used for modification, e.g. insertion or deletion of a marker gene such as GFP or lacZ from the parental virus at the intended insertion site. In a most preferred embodiment the insertion site is the ICP34.5 locus of HSV, and therefore the plasmid used for manipulation contains HSV sequences flanking this insertion site, between which are an expression cassette encoding GM-CSF and the immune co-stimulatory pathway activating molecule. In this case, the parental virus may contain a cassette encoding GFP in place of ICP34.5 and recombinant virus plaques are selected through the loss of expression of GFP. In a most preferred embodiment the US11 gene of HSV is also expressed as an IE gene. This may be accomplished through deletion of the ICP47-encoding region, or by other means.

The GM-CSF encoding sequences and immune co-stimulatory pathway activating molecule encoding sequences are inserted into the viral genome under appropriate regulatory control. This may be under the regulatory control of natural promoters of the virus species of the invention used, depending on the species and insertion site, or preferably under the control of heterologous promoters. Suitable heterologous promoters include mammalian promoters, such as the IEF2a promoter or the actin promoter. More preferred are strong viral promoters such as the CMV IE promoter, the RSV LTR, the MMLV LTR, other retroviral LTR promoters, or promoters derived from SV40. Preferably each exogenous gene (e.g. encoding the GM-CSF and immune co-stimulatory pathway activating molecule) will be under separate promoter control, but may also be expressed from a single RNA transcript, for example through insertion of an internal ribosome entry sites (IRES) between protein coding sequences. RNA derived from each promoter is typically terminated using a polyadenylation sequence (e.g. mammalian sequences such as the bovine growth hormone (BGH) poly A sequence, synthetic polyadenylation sequences, the rabbit betaglobin polyadenylation sequence, or viral sequences such as the SV40 early or late polyadenylation sequence).

### Pharmaceutical Compositions

The invention provides a pharmaceutical composition comprising the virus and a pharmaceutically acceptable carrier or diluent. Suitable carriers and diluents include isotonic saline solutions, for example phosphate-buffered saline. The composition may further comprise other constituents such as sugars or proteins to improve properties such as stability of the product. Alternatively a lyophilized formulation may be used, which is reconstituted in a pharmaceutically acceptable carrier or diluent before use.

The choice of carrier, if required, is frequently a function of the route of delivery of the composition. Within this invention, compositions may be formulated for any suitable route and means of administration. Pharmaceutically acceptable carriers or diluents are those used in compositions suitable for intra-tumoral administration, intravenous/intraarterial administration, administration into the brain or administration into a body cavity (e.g. bladder, pleural cavity or by intraperitoneal administration). The composition may be administered in any suitable form, preferably as a liquid.

The present invention also provides a product of manufacture comprising a virus of the invention in a sterile vial, ampoule or syringe.

### Medical Uses/Methods of Treatment

The invention provides the virus of the invention for use in the treatment of the human or animal body by therapy, particularly for use in a method of treating cancer. The cancer is typically in a mammal, preferably in a human. The virus kills infected tumour cells by lysis and by causing infected tumor cells to fuse with one another. The virus of the invention also elicits a systemic anti-tumor immune response, augmented through the expression of GM-CSF and the immune co-stimulatory pathway activating molecule, which also kills cancer cells.

The invention also provides a method of treating cancer, the method comprising administering a therapeutically effective amount of the virus of the invention to an individual in need thereof.

The invention additionally provides the use of the virus of the invention in the manufacture of a medicament for treating cancer.

The virus of the invention is particularly useful in treating any solid tumor including any adenocarcinoma, carcinoma, melanoma or sarcoma. For example, the virus of the invention is useful in treating head and neck, prostate, breast, ovarian, lung, liver, endometrial, bladder, gall bladder, pancreas, colon, kidney, stomach/gastric, esophageal, or cervical cancers, mesothelioma, melanoma or other skin cancer, lymphoma, glioma or other cancer of the nervous system, or sarcomas such as soft tissue sarcoma.

The virus of the invention may be used to treat malignant tumors, including tumors that have metastasised from the site of the original tumor. In this embodiment, the virus may be administered to the primary tumor or to one or more secondary tumors.

The virus of the invention may be administered in combination with other therapeutic agents, including chemotherapy, targeted therapy, immunotherapy (including immune checkpoint blockade, i.e. administration of one or more antagonist of an immune co-inhibitory pathway, and/or one or more agonist of an immune co-stimulatory pathway) and/or in combination with radiotherapy and/or in combination with any combination of these. The therapeutic agent is preferably an anti-cancer agent.

The virus of the invention may be administered in combination with a second virus, such as a second oncolytic virus.

For example, the therapeutic agent may comprise an immunogen (including a recombinant or naturally occurring antigen, including such an antigen or combination of antigens delivered as DNA or RNA in which it/they are encoded), to further stimulate an immune response, such as a cellular or humoral immune response, to tumor cells, particularly tumor neoantigens. The therapeutic agent may be an agent intended to increase or potentiate an immune response, such as a cytokine, an agent intended to inhibit an immune checkpoint pathway or stimulate an immune potentiating pathway or an agent which inhibits the activity of regulatory T cells (Tregs) or myeloid derived suppressor cells (MDSCs).

The therapeutic agent may be an agent known for use in an existing cancer therapeutic treatment. The therapeutic agent may be radiotherapy or a chemotherapeutic agent. The therapeutic agent may be selected from cyclophosmamide, alkylating-like agents such as cisplatin or melphalan, plant alkaloids and terpenoids such as vincristine or paclitaxel (Taxol), antimetabolites such as 5-fluorouracil, topoisomerase inhibitors type I or II such as camptothecin or doxorubicin, cytotoxic antibiotics such as actinomycin, anthracyclines such as epirubicin, glucocorticoids such as triamcinolone, inhibitors of protein, DNA and/or RNA synthesis such as methotrexate and dacarbaxine, histone deacetylase (HDAC) inhibitors, or any other chemotherapy agent.

The therapeutic agent may be one, or a combination of: immunotherapeutics or immunomodulators, such as TLR agonists; agents that down-regulate T-regulatory cells such as cyclophosphamide; or agents designed to block immune checkpoints or stimulate immune potentiating pathways, including but not limited to monoclonal antibodies, such as a CTLA-4 inhibitor, a PD-1 inhibitor, a PD-L1 inhibitor, a LAG-3 inhibitor, a TIM-3 inhibitor, a VISTA inhibitor, a CSFIR inhibitor, an IDO inhibitor, a CEACAM1 inhibitor, a GITR agonist, a 4-1-BB agonist, a KIR inhibitor, a SLAMF7 inhibitor, an OX40 agonist, a CD40 agonist, an ICOS agonist or a CD47 inhibitor. In a preferred embodiment, the therapeutic agent is a CTLA-4 inhibitor such as an anti-CTLA-4 antibody, a PD1 inhibitor, such as an anti-PD-1 antibody or a PD-L1 inhibitor such as an anti-PD-L1 antibody. Such inhibitors, agonists and antibodies can be generated and tested by standard methods known in the art.

Immunotherapeutic agents may also include bi-specific antibodies, cell based-therapies based on dendritic cells, NK cells or engineered T cells such CAR-T cells or T cells expressing engineered T cell receptors. Immunotherapeutic agents also include agents that target a specific genetic mutation which occurs in tumors, agents intended to induce immune responses to specific tumor antigens or combinations of tumor antigens, including neoantigens and/or agents intended to activate the STING/cGAS pathway, TLR or other innate immune response and/or inflammatory pathway, including intra-tumoral agents.

For example, a virus of the invention may be used: in combination with dacarbazine, a BRAF inhibitor and or CTLA-4, PD1 or PD-L1 blockade to treat melanoma; in combination with taxol, doxorubicin, vinorelbine, cyclophosphamide and/or gemcitabine to treat breast cancer; in combination with 5-fluorouracil and optionally leucovorin, irinoteacan and/or oxaliplatin to treat colorectal cancer; in combination with taxol, carboplatin, vinorelbine and/or gemcitabine, PD-1 or PD-L1 blockade to treat lung cancer; in combination with cisplatin and/or radiotherapy to treat head and neck cancer.

The therapeutic agent may be an inhibitor of the idoleamine 2,3-dioxygenase (IDO) pathway. Examples of IDO inhibitors include epacadostat (INCB024360), 1-methyl-tryptophan, Indoximod (1-methyly-D-tryptophan), GDC-0919 or F001287.

The mechanism of action of IDO in suppressing anti-tumor immune responses may also suppress immune responses generated following oncolytic virus therapy. IDO expression is induced by toll like receptor (TLR) activation and interferon-γ both of which may result from oncolytic virus infection. One embodiment of the use of oncolytic virus therapy for cancer treatment includes combination of an oncolytic virus, including a virus expressing GM-CSF and an immune co-stimulatory pathway activating molecule or molecules with an inhibitor of the IDO pathway and optionally one or more antagonist of an immune co-inhibitory pathway and/or one or more agonist of an immune co-stimulatory pathway, including those targeting CTLA-4, PD-1 and/or PD-L1.

The invention also provides a method of treating cancer, which comprises administering a therapeutically effective amount of an oncolytic virus, an inhibitor of the indoleamine 2,3-dioxygenase (IDO) pathway and a further antagonist of an immune co-inhibitory pathway, and/or an agonist of an immune co-stimulatory pathway to a patient in need thereof.

The oncolytic virus is preferably a modified clinical isolate. The oncolytic virus is preferably a pox virus, more preferably a HSV, such as a HSV1 and/or a HSV rendered functionally inactive for ICP34.5 and/or ICP47.

The oncolytic virus may express an immune stimulating molecule, such as GM-CSF and/or a co-stimulatory pathway encoding molecule such as CD4OL, GITRL, OX4OL, 4-I-BBL or ICO5L, and/or an inhibitor of CTLA-4, and/or a fusogenic protein, such as the GALV fusogenic glycoprotein with the R sequence mutated or deleted.

The further antagonist of an immune co-inhibitory pathway is preferably an antagonist of CTLA-4, an antagonist of PD1 or an antagonist of PD-L1. For example, the further antagonist of an immune co-inhibitory pathway may be an inhibitor of the interaction between PD1 and PD-L1.

Where a therapeutic agent and/or radiotherapy is used in conjunction with a virus of the invention, administration of the virus and the therapeutic agent and/or radiotherapy may be contemporaneous or separated by time. The composition of the invention may be administered before, together with or after the therapeutic agent or radiotherapy. The method of treating cancer may comprise multiple administrations of the virus of the invention and/or of the therapeutic agent and/or radiotherapy. In preferred embodiments, in the case of combination with immune checkpoint blockade or other immune potentiating agents, the virus of the invention is administered once or multiple times prior to the concurrent administration of the immune checkpoint blockade or other immune potentiating agent or agents thereafter, or concurrent with the administration of the immune checkpoint blockade or other immune potentiating agent or agents without prior administration of the virus of the invention.

The virus of the invention may be administered to a subject by any suitable route. Typically, a virus of the invention is administered by direct intra-tumoral injection. Intra-tumoral injection includes direct injection into superficial skin, subcutaneous or nodal tumors, and imaging guided (including CT, MRI or ultrasound) injection into deeper or harder to localize deposits including in visceral organs and elsewhere. The virus may be administered into a body cavity, for example into the pleural cavity, bladder or by intra-peritoneal administration. The virus may be injected into a blood vessel, preferably a blood vessel supplying a tumor.

Therapeutic agents which may be combined with a virus of the invention can be administered to a human or animal subject in vivo using a variety of known routes and techniques. For example, the composition may be provided as an injectable solution, suspension or emulsion and administered via parenteral, subcutaneous, oral, epidermal, intradermal, intramuscular, interarterial, intraperitoneal, intravenous injection using a conventional needle and syringe, or using a liquid jet injection system. The composition may be administered topically to skin or mucosal tissue, such as nasally, intratrachealy, intestinally, sublingually, rectally or vaginally, or provided as a finely divided spray suitable for respiratory or pulmonary administration. In preferred embodiments, the compositions are administered by intravenous infusion, orally, or directly into a tumor.

The virus and/or therapeutic agent may be administered to a subject in an amount that is compatible with the dosage composition that will be therapeutically effective. The administration of the virus of the invention is for a “therapeutic” purpose. As used herein, the term “therapeutic” or “treatment” includes any one or more of the following as its objective: the prevention of any metastasis or further metastasis occurring; the reduction or elimination of symptoms; the reduction or complete elimination of a tumor or cancer, an increase in the time to progression of the patient's cancer; an increase in time to relapse following treatment; or an increase in survival time.

Therapeutic treatment may be given to Stage I, II, III, or IV cancers, preferably Stage II, III or IV, more preferably Stage III or IV, pre- or post-surgical intervention (i.e. following recurrence or incomplete removal of tumors following surgery), preferably before any surgical intervention (either for resection of primary or recurrent/metastatic disease), or following recurrence following surgery or following incomplete surgical removal of disease, i.e. while residual tumor remains.

Therapeutic treatment may be carried out following direct injection of the virus composition into target tissue which may be the tumor, into a body cavity, or a blood vessel. As a guide, the amount of virus administered is in the case of HSV in the range of from 10⁴ to 10¹⁰ pfu, preferably from 10⁵ to 10⁹ pfu. In the case of HSV, an initial lower dose (e.g. 10⁴ to 10⁷ pfu) may be given to patients to seroconvert patients who are seronegative for HSV and boost immunity in those who are seropositive, followed by a higher dose then being given thereafter (e.g. 10⁶ to 10⁹ pfu). Typically up to 20 ml of a pharmaceutical composition consisting essentially of the virus and a pharmaceutically acceptable suitable carrier or diluent may be used for direct injection into tumors, or up to 50 ml for administration into a body cavity (which may be subject to further dilution into an appropriate diluent before administration) or into the bloodstream. However for some oncolytic therapy applications larger or smaller volumes may also be used, depending on the tumor and the administration route and site.

The routes of administration and dosages described are intended only as a guide since a skilled practitioner will be able to determine readily the optimum route of administration and dosage. The dosage may be determined according to various parameters, especially according to the location of the tumor, the size of the tumor, the age, weight and condition of the patient to be treated and the route of administration. Preferably the virus is administered by direct injection into the tumor or into a body cavity. The virus may also be administered by injection into a blood vessel. The optimum route of administration will depend on the location and size of the tumor. Multiple doses may be required to achieve an immunological or clinical effect, which, if required, will be typically administered between 2 days to 12 weeks apart, preferably 3-days to 3 weeks apart. Repeat doses up to 5 years or more may be given, preferably for up to one month to two years dependent on the speed of response of the tumor type being treated and the response of a particular patient, and any combination therapy which may also be being given.

The following Examples illustrate the invention.

### Example 1. Construction of a Virus of the Invention

The virus species used to exemplify the invention is HSV, specifically HSV1. The strain of HSV1 used for exemplification is identified through the comparison of more than 20 primary clinical isolates of HSV1 for their ability to kill a panel of human tumor-derived cell lines and choosing the virus strain with the greatest ability to kill a broad range of these rapidly, and at low dose. Tumor cell lines used for this comparison include U87MG (glioma), HT29 (colorectal), LNCaP (prostate), MDA-MB-231 (breast), SK-MEL-28 (melanoma), Fadu (squamous cell carcinoma), MCF7 (breast), A549 (lung), MIAPACA-2 (pancreas), CAPAN-1 (pancreas), and/or HT1080 (fibrosarcoma).

Specifically, each primary clinical isolate of HSV is titrated onto each of the cell lines used for screening at MOIs such as 1, 0.1, 0.01 and 0.001 and assessed for the extent of cell death at time points such as 24 and 48 hrs at each dose. The extent of cell killing may be assessed by e.g. microscopic assessment of the proportion of surviving cells at each time point, or e.g. a metabolic assay such as an MTT assay. The exemplary virus of the invention is then constructed by deletion of ICP47 from the viral genome using homologous recombination with a plasmid containing regions flanking HSV1 nucleotides 145300 to 145582 (HSV1 nucleotides 145300 to 145582 being the sequences to be deleted; HSV1 strain 17 sequence Genbank file NC 001806.2) between which are encoded GFP. GFP expressing virus plaques are selected, and GFP then removed by homologous recombination with the empty flanking regions and plaques which do not express GFP are selected. This results in an ICP47 deleted virus in which US11 is expressed as an IE protein as it is now under the control of the ICP47 promoter. ICP34.5 is then deleted using homologous recombination with a plasmid containing regions flanking HSV1 nucleotides 124953 to 125727 (HSV1 nucleotides 124953 to 125727 being the sequences to be deleted; HSV1 strain 17 sequence Genbank file NC_001806.2) between which GFP is encoded. GFP expressing virus plaques are again selected, and GFP then removed by homologous recombination with the same flanking regions but between which are now an expression cassette comprising a codon optimized version of the mouse GM-CSF sequence, a codon optimized version of the GALV R-sequence and codon optimized version of mouse soluble multimeric CD40L driven by a CMV, an RSV and an SV40 promoter. Non-GFP expressing plaques are selected.

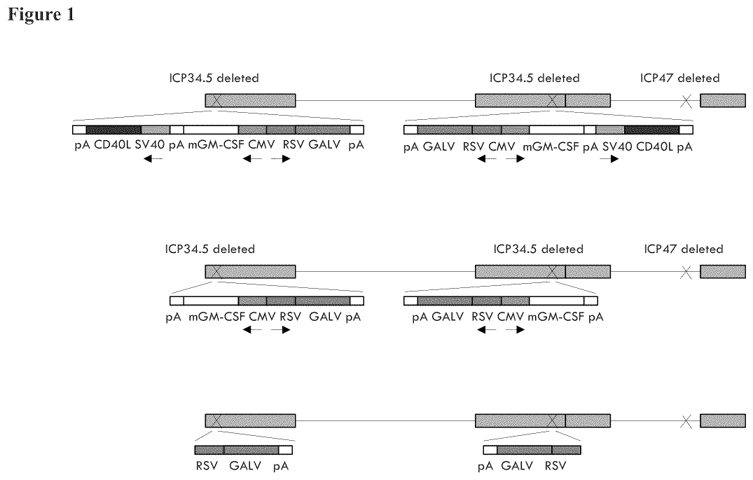

The structure of the resulting virus is shown in FIG. 1. The mGM-CSF, CD40L and GALV-R-sequences are shown in SEQ ID NOs 2, 14 and 8 respectively. The structure of the resulting virus is confirmed by restriction digestion and Southern blot, GM-CSF and CD40L expression is confirmed by ELISA, and GALV-R-expression is confirmed by infection of human HT1080 tumor cells and the observation of syncitial plaques.

Viruses are also constructed using similar procedures which only have inserted the gene for GALVR-or mouse GM-CSF and GALV-R-, but without CD40L. The structures of these viruses are also shown in FIG. 1.

For human use, hGM-CSF and hCD40L are used, the sequence for codon optimised versions of which are shown in SEQ ID NO 4 and 13.

### Example 2. The Effect of the Combined Expression of GM-CSF and an Immune Co-Stimulatory Pathway Activating Molecule from an Oncolytic Virus in Mouse Tumor Models

The GALV R-protein causes cell to cell fusion in human cells but not in mouse cells because the PiT-1 receptor required for cell fusion to occur has a sequence in mice which does not allow cell fusion to occur. As a result mouse tumor cells expressing human PiT-1 are first prepared using methods standard in the art. Human PiT-1 is cloned into a lentiviral vector also comprising a selectable marker gene. The vector is transfected into target CT26 mouse colorectal cancer tumor cells and clones resistant to the selectable marker are selected to generate CT26/PiT-1 cells. PiT-1 expression is confirmed by western blotting in untransfected cells and in cells transfected with the PiT-1 expressing lentivirus and by transfection of a plasmid expressing GALV-R-and confirmation that cell fusion occurs.

The utility of the invention is demonstrated by administering CT26/PiT-1 cells into both flanks of Balb/c mice and allowing the CT26/PiT-1 tumors to grow to approximately 0.5 cm in diameter.

The following treatments are then administered to groups of mice (five per group), into one flank of each mouse only 3 times per week for two weeks:
50 μl of saline (1 group);
50 μl of 10⁵ pfu/ml, 10⁶ pfu/ml, or 10⁷ pfu/ml of the HSV with only GALVR-inserted (3 groups);
50 μl of 10⁵ pfu/ml, 10⁶ pfu/ml, or 10⁷ pfu/ml of the HSV with only GALVR-and mouse GM-CSF inserted (3 groups);
50 μl of 10⁵ pfu/ml, 10⁶ pfu/ml, or 10⁷ pfu/ml of the virus with GALVR- and both mouse GM-CSF and CD40L inserted (3 groups).

Effects on tumor growth are then observed for up to one month. Superior tumor control and shrinkage in both injected and uninjected tumors with the virus expressing GM-CSF and CD40L as compared to the other groups is observed, including through an improved dose response curve.

Example 3. The Effect of Combined Expression of GM-CSF and an Immune Co-Stimulatory Pathway Activating Molecule from an Oncolytic Virus on the Therapeutic Effect of Immune Checkpoint Blockade in Mouse Tumor Models

The experiment in Example 2 above is repeated but mice are additionally dosed bi-weekly by the intra-peritoneal route with an antibody targeting mouse PD-1 (10 mg/kg; Bioxcell RMP-1-14 on the same days as virus dosing) or an antibody targeting mouse CTLA-4 (10 mg/kg; Bioxcell 9H10 on the same days as virus dosing). An additional group of mice is added which receive no antibody treatment. More specifically, groups of mice receive (1) saline, (2) HSV with GALVR-inserted as in Example 2, (3) HSV with GM-CSF and GALV-R-inserted as in Example 2, (4) HSV with GM-CSF, CD40L and GALV-R-inserted as in Example 2, (5) PD-1 antibody, (6) CTLA-4 antibody, (7) HSV with GALV-R-inserted plus PD-1 antibody, (8) HSV with GALV-R-inserted gene plus CTLA-4 antibody, (9) HSV with GM-CSF and GALV-R- and PD-1 antibody or (10) HSV with GM-CSF and GALV-R-and CTLA-4 antibody (11) HSV with GM-CSF, CD40L and GALV-R- and PD-1 antibody or (12) HSV with GM-CSF, CD40L and GALV-R- and CTLA-4 antibody. Superior tumor control and shrinkage in both injected and uninjected tumors with the virus expressing GM-CSF and CD40L together with the anti-PD-1 antibody or the anti-CTLA-4 antibody as compared to the other groups is observed, including through an improved dose response curve.

### Example 4. Collection of Clinical Isolates

The virus species used to exemplify the invention is HSV, specifically HSV1. To exemplify the invention, 181 volunteers were recruited who suffered from recurrent cold sores. These volunteers were given sample collection kits (including Sigma Virovult collection tubes), and used these to swab cold sores when they appeared following which these samples were shipped to Replimune, Oxford UK. From June 2015-February 2016, swabs were received from 72 volunteers. A sample of each swab was used to infect BHK cells. Of these 36 live virus samples were recovered following plating out and growth on BHK cells. These samples are detailed in Table 1.

TABLE 1
Details of Tested Swab Samples & Result

| Sample Number | Virus retrieved |
|---|---|
| RH001A | No |
| RH001B | |
| RH002A | Yes |
| RH003A | No |
| RH004A | Yes |
| RH004B | |
| RH005A | No |
| RH005B | |
| RH006A | No |
| RH006B | |
| RH007A | Yes |
| RH007B | |
| RH007C | |
| RH008A | No |
| RH008B | |
| RH008C | |
| RH009A | No |
| RH009B | |
| RH010A | No |
| RH011A | No |
| RH011B | |
| RH011C | |
| RH012A | No |
| RH013A | No |
| RH014A | Yes |
| RH014B | |
| RH015A | Yes |
| RH016A | No |
| RH016B | |
| RH017A | Yes |
| RH018A | Yes |
| RH018B | |
| RH018C | |
| RH019A | No |
| RH019B | |
| RH019C | |
| RH020A | Yes - RH020A only |
| RH020B | |
| RH020C | |
| RH021A | Yes |
| RH021B | |
| RH022A | Yes |
| RH022B | |
| RH023A | Yes |
| RH024A | No |
| RH025A | Yes - RH025B only |
| RH025B | |
| RH026A | Yes |
| RH027A | No |
| RH027B | |
| RH027C | |
| RH028A | No |
| RH028B | |
| RH028C | |
| RH029A | No |
| RH030A | No |
| RH031A | Yes - RH031A to RH031D |
| RH031B | |
| RH031C | |
| RH031D | |
| RH031E | |
| RH031F | |
| RH032A | No |
| RH033A | No |
| RH033B | |
| RH033C | |
| RH034A | No |
| RH034B | |
| RH034C | |
| RH035A | No |
| RH036A | Yes |
| RH037A | Yes |
| RH038A | Yes |
| RH039A | No |
| RH039B | |
| RH039C | |
| RH040A | Yes |
| RH040B | |
| RH040C | |
| RH041A | Yes |
| RH042A | Yes |
| RH043A | No |
| RH043B | |
| RH043C | |
| RH044A | No |
| RH045A | No |
| RH046A | Yes |
| RH047A | Yes - RH047A and RH047C |
| RH047B | |
| RH047C | |
| RH048A | No |
| RH049A | No |
| RH049B | |
| RH049C | |
| RH050A | No |
| RH051A | Yes |
| RH051B | |
| RH052A | Yes - RH052A only |
| RH052B | |
| RH053A | No |
| RH054A | No |
| RH055A | No |
| RH055B | |
| RH056A | Yes |
| RH057A | No |
| RH058A | Yes |
| RH058B | |
| RH059A | No |
| RH060A | No |
| RH061A | Yes |
| RH062A | No |
| RH063A | No |
| RH064A | Yes |
| RH065A | Yes |
| RH065B | |
| RH066A | No |
| RH067A | No |
| RH067B | |
| RH068A | No - contaminated |
| RH069A | No |
| RH069A | |
| RH070A | Yes |
| RH071A | Yes |
| RH072A | No |
| RH073A | Yes |
| RH073B | |
| RH074A | No |
| RH074B | |
| RH075A | No |
| RH076A | No |
| RH078A | No |
| RH078B | |
| RH079B | Yes |
| RH079B | |
| RH080A | No |
| RH081A | Yes |
| RH082A | No |
| RH082B | |
| RH083A | Yes |
| RH083B | |
| RH084A | Yes |
| RH084B | |
| RH084C | |
| RH085A | No |
| RH086A | No |
| RH087A | Yes - RH078B only |
| RH087B | |

Designations A, B, C etc. indicate multiple swabs from the same volunteer.

### Example 5. Identification of Clinical Isolates with Improved Anti-Tumor Effects

The abilities of the primary clinical isolates of HSV1 to kill a panel of human tumor-derived cell lines was tested. The tumor cell lines used for this comparison were HT29 (colorectal), MDA-MB-231 (breast), SK-MEL-28 (melanoma), Fadu (squamous cell carcinoma), MCF7 (breast), A549 (lung), MIAPACA-2 (pancreas) and HT1080 (fibrosarcoma). The cell lines were used to test for the level of CPE achieved at a range of MOI and times post infection for each of the primary clinical isolates.

Experiments were conducted in parallel using 5 to 8 of the new viruses strains at the same time. The virus strains were plated out in duplicate at a range of MOIs (0.001-1), and the extent of CPE following crystal violet staining was assessed at 24 and 48 hours following infection. The viral strains which were most effective at killing the tumor cell lines were scored, and the most effective two or three strains from each screen of 5-8 strains were identified and compared in parallel in a further experiment to identify the top strains for further development.

The initial screens demonstrated substantial variability in the ability of the different strains to kill the different tumor cell lines. Of an initial 29 strains tested, 8 strains of interest were identified in the initial screens for further comparison. These were strains RH004A, RH015A, RH018A, RH021A, RH023A, RH31A, RH040A, and RH047A.

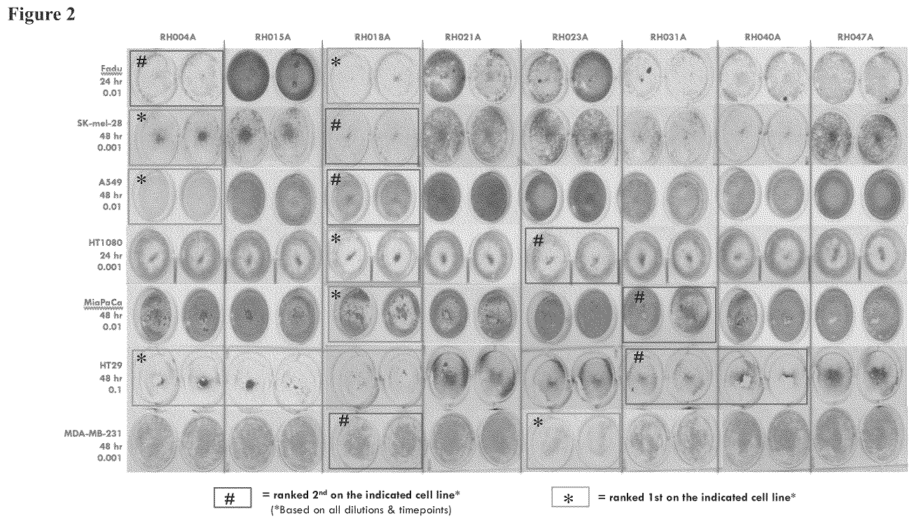

The 8 strains for further comparison were tested in parallel on the panel of tumor cell lines, and their relative ability to kill these tumor cell lines was assessed following crystal violet staining and observation for CPE. FIG. 2 shows a representative time point and MOI for these viruses on each of the viruses on each of the cell lines demonstrating the differential ability of the viruses to kill the target tumor cell lines observed.

There was substantial variation amongst the strains, and it was found that while a particular strain may be particularly effective at killing one cell line, it is not necessarily particularly effective at killing other cell lines too, further demonstrating the degree of variability in the ability of clinical strains of HSV to kill tumor cells of different types.

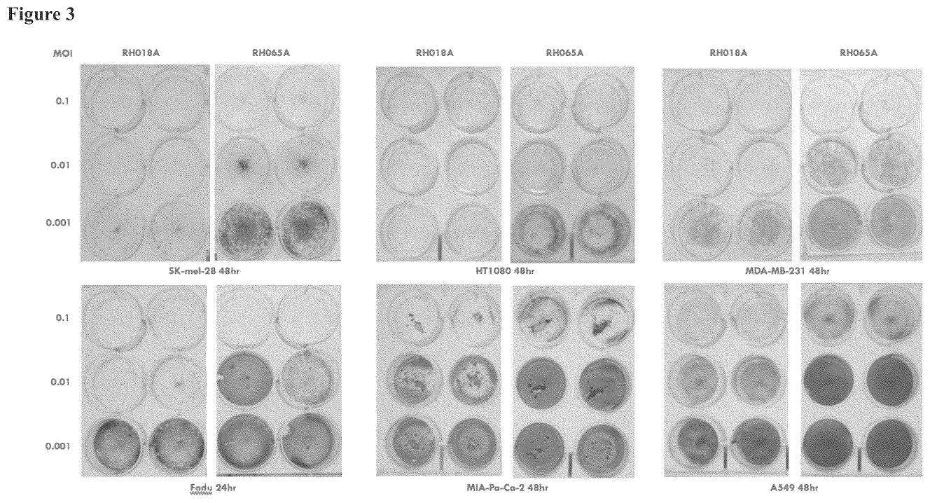

FIG. 3 also indicates which of the virus strains was both best and second best at killing each of the cell lines, enabling the virus strains to be rank ordered as to their overall relative ability to kill the panel of cell lines as a whole. This analysis demonstrated that strains RH004A, RH015A, RH018A, RH031A and RH040A were relatively more effective than the other strains, and these five strains were chosen for potential further development as oncolytic agents. Of these top five strains, the relative rank order based on their abilities to kill across the panel of cell lines was RH018A>RH004A>RH031A>RH040A>RH015A.

More specifically, in these experiments, the tumor cell lines were used to seed multi-well tissue culture plates so that they were about 80% confluent on the day of infection. Representative wells from each tumor cell line were trypsinised and the number of cells in the well determined. These cell counts are used to determine the volume of each clinical isolate required to give an MOI of 1, 0.1, 0.01 and 0.001. Separate wells of a tumor cell line were infected with the clinical isolate at these MOI. All infections are carried out in quadruplicate. Duplicate wells were incubated for 24 hours and duplicate wells were incubated for 48 hours, both at 37° C., 5% CO₂, prior to fixation of the cells with glutaraldehyde and staining with crystal violet. The level of cell lysis was then assessed by gross observation, microscopy (cell counts) and photography.

Strain RH018A, the strain ranked first of all the strains tested was compared to an ‘average’ strain from the screen (i.e. a strain which was not in the top 8, but was also not in the group of strains which were least effective and killing the panel of tumor cell lines). This comparison showed that Strain RH018A was approximately 10 fold more effective than this average strain (Strain RH06SA) at killing the tumor cell lines (i.e. approximately 10 fold less of Strain RH018A was needed to kill an equal proportion of cells than was needed of Strain RH065A). This is shown in FIG. 3.

### Example 6. Modification of Clinical Isolates

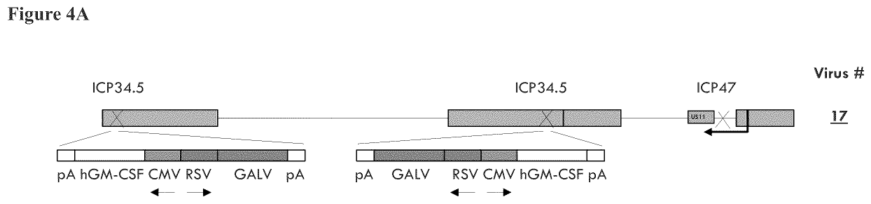

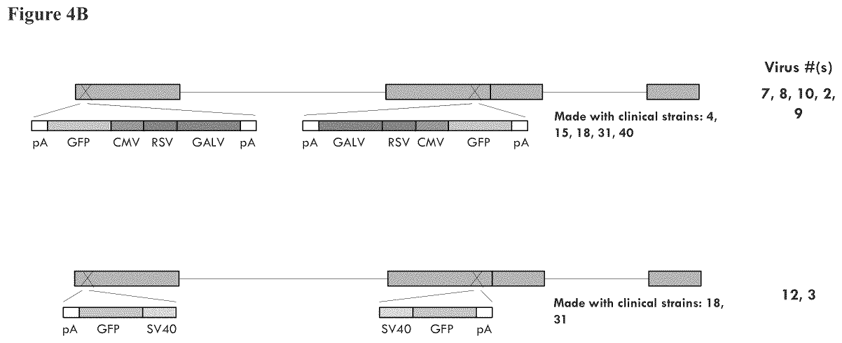

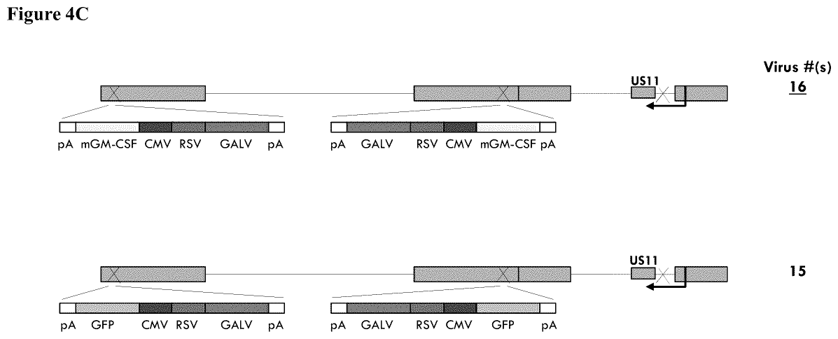

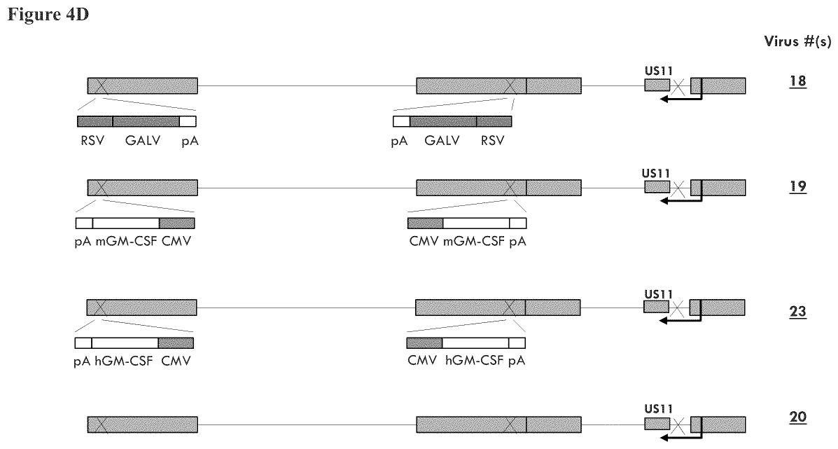

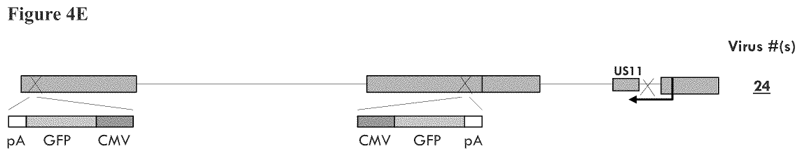

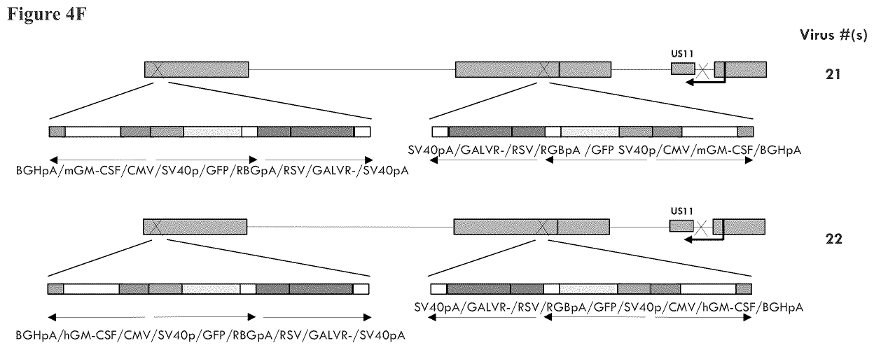

In this Example the clinical isolates selected in Example 5 were modified by deletion of ICP34.5 from the viral genome using homologous recombination with a plasmid containing regions flanking the ICP34.5 encoding gene (nucleotides 143680-145300 and 145,582-147,083; HSV1 strain 17 sequence Genbank file NC_001806.2) between which are encoded GFP and the GALV-R-fusogenic glycoprotein. The structure of this virus, (Virus 10) is shown in FIGS. 4A-4F.

Additional viruses based on Strain RH018A were also constructed in which both ICP34.5 and ICP47 (using flanking regions containing nucleotides 123464-124953 and 125727-126781; HSV1 strain 17 sequence Genbank file NC_001806.2) were deleted (resulting in placement of US11 under the control of the ICP47 promoter). To construct these viruses, GFP expressing virus plaques, with GFP expressed in place of ICP47 were first selected. GFP was then removed by homologous recombination with the empty flanking regions, and plaques not expressing GFP were selected. This resulted in an ICP47 deleted virus in which US11 is expressed as an IE protein as it is now under the control of the ICP47 promoter. ICP34.5 was then deleted using homologous recombination with a plasmid containing regions flanking HSV1 nucleotides 143680-145300 and 145,582-147,083; HSV1 strain 17 sequence Genbank file NC_001806.2) between which GFP is encoded. GFP expressing virus plaques were again selected, and GFP then removed by homologous recombination with the same flanking regions but between which are now an expression cassette comprising the genes to be inserted. The viruses that were constructed are shown in FIGS. 1, 4 and 5. These included a codon optimized version of the mouse GM-CSF sequence and a codon optimized version of the GALV R-sequence driven by the CMV IE promoter and RSV promoter respectively, in a back to back orientation and again selecting virus plaques which do not express GFP. This virus construction was performed using methods which are standard in the art.

The mGM-CSF and GALV-R-sequences are shown in SEQ ID NOs 2 and 8 respectively. The structure of the resulting virus was confirmed by PCR, GM-CSF expression was confirmed by ELISA, and GALV-R-expression was confirmed by infection of human HT1080 tumor cells and the observation of syncitial plaques.

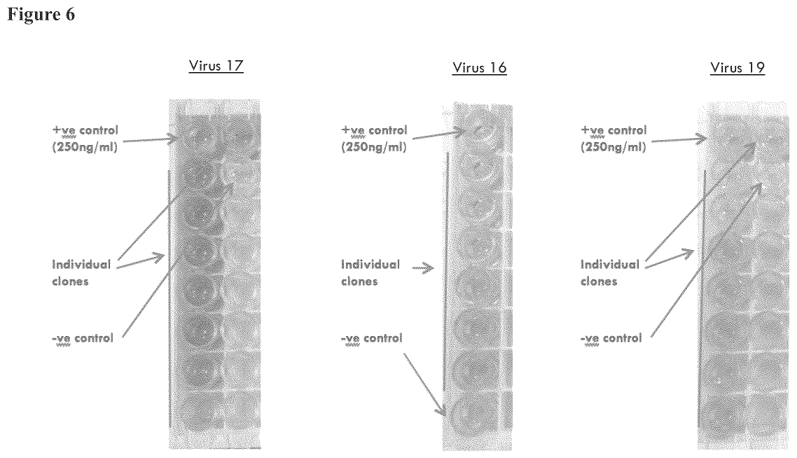

For human use, hGM-CSF is used, the sequence for a codon optimised version of which is shown in SEQ ID NO 4. The structure of this virus is shown in FIGS. 4A-4F. Expression of mouse or human GM-CSF from viruses 16, 17 and 19 is shown in FIG. 6.

Example 7. A virus of the Invention Modified for Oncolytic use and Expressing a Fusogenic Glycoprotein Shows Enhanced Tumor Cell Killing in vitro as Compared to a Virus which does not express a Fusogenic Glycoprotein

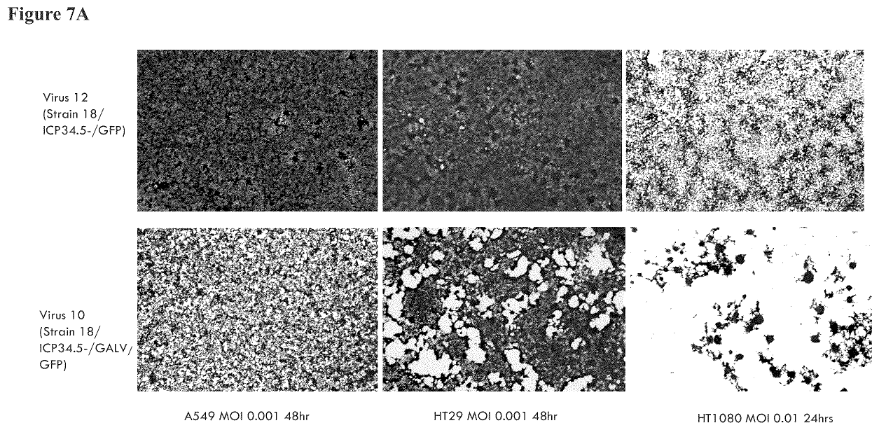

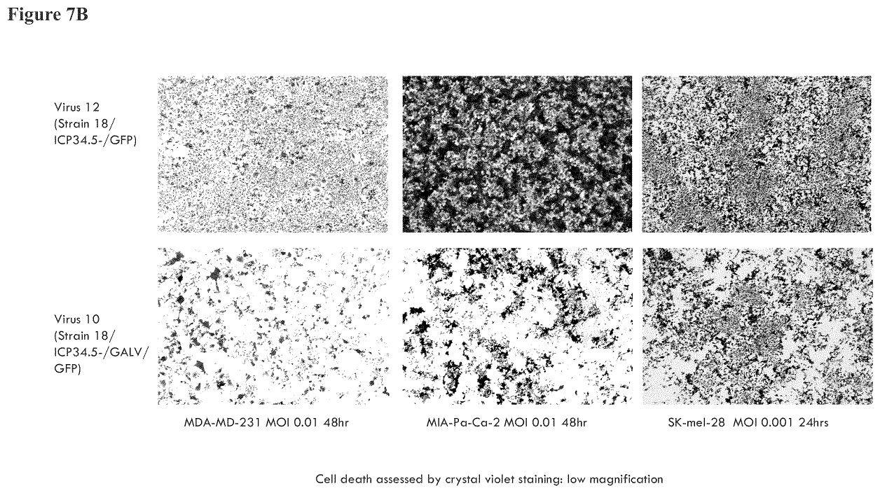

Virus 10 (see FIGS. 4A-4F), based on clinical Strain RH018A in which ICP34.5 is deleted and which expresses GALVR-and GFP, was compared in vitro to a virus which expresses only GFP (Virus 12). Virus 10 showed enhanced killing on a panel of human tumor cell lines as compared to Virus 12, as shown in FIGS. 7A-7B.

### Example 8. A Virus of the Invention Modified for Oncolytic Use Shows Enhanced Tumor Cell Killing as Compared to a Similarly Modified Virus which is not of the Invention

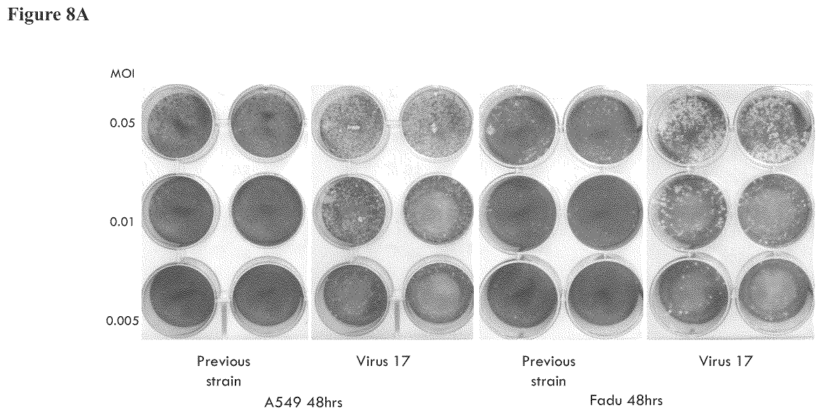

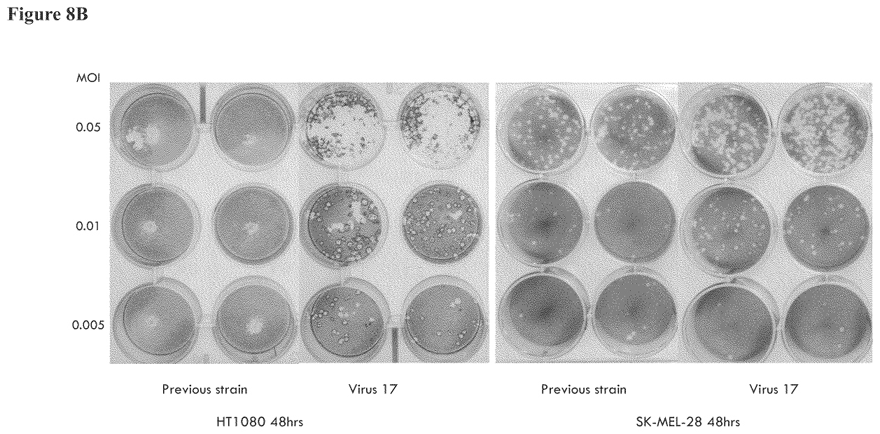

Virus 17 (see FIGS. 4A-4F), based on clinical Strain RH018A in which ICP34.5 and ICP47 are deleted and which expresses GALVR-and GM-CSF, was compared in vitro to a known virus which was also deleted for ICP34.5 and ICP47 but which was not derived from a strain of the invention and which expresses only GM-CSF. Virus 17 showed enhanced killing on a panel of human tumor cell lines as compared to the previous virus, as shown in FIGS. 8A-8B.

### Example 9. A Virus of the Invention Modified for Oncolytic Use Effectively Treats Mouse Tumors in Vivo

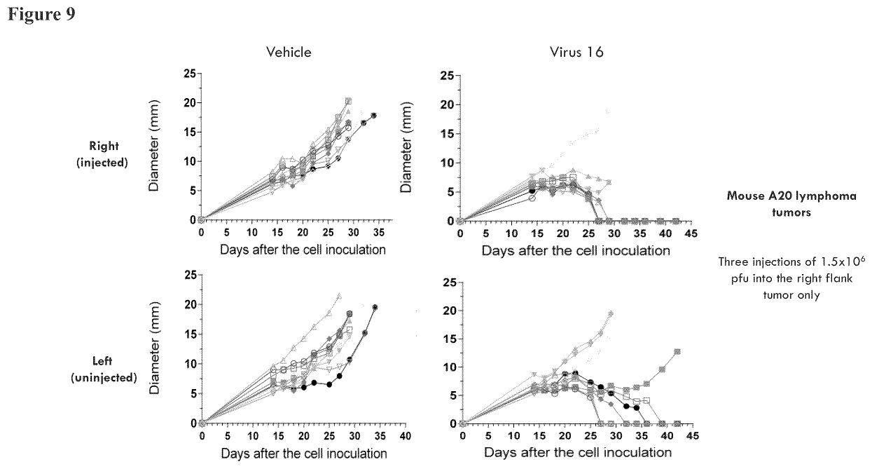

Virus 16 was tested in mice harboring A20 lymphoma tumors in the left and right flanks. One million tumor cells were first implanted in both flanks of Balb/c mice and tumors allowed to grow to 0.5-0.7 cm in diameter. Tumors on the right flank were then injected 3 times (every other day) with either vehicle (10 mice) or 5×10exp6 pfu of Virus 16 (10 mice), and effects on tumor size observed for a further 30 days. This demonstrated that both injected and uninjected tumors were effectively treated with Virus 16 (see FIG. 9).

### Example 10. The Effect of the Combined Expression of a Fusogenic Protein and an Immune Stimulatory Molecule from an Oncolytic Virus of the Invention in a Rat Tumor Model

The GALV R-protein causes cell to cell fusion in human cells but not in mouse cells. However, GALV R-does cause fusion in rat cells.

The utility of the invention was further demonstrated by administering 9L cells into the flanks of Fischer 344 rats and allowing the 9L tumors to grow to approximately 0.5 cm in diameter.

The following treatments were then administered to groups of rats (ten per group), into one flank only of each rat three times per week for three weeks:
50 μl of vehicle;
50 μl of 10⁷ pfu/ml of Virus 19 (expresses mGM-CSF but not GALV R-);
50 μl of 10⁷ pfu/ml of Virus 16 (expresses both mouse GM-CSF and GALV-R-).

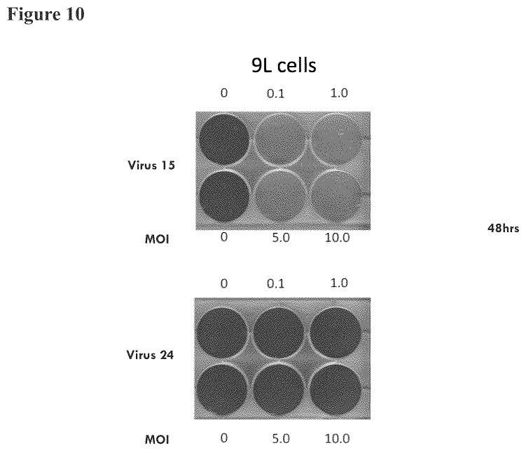

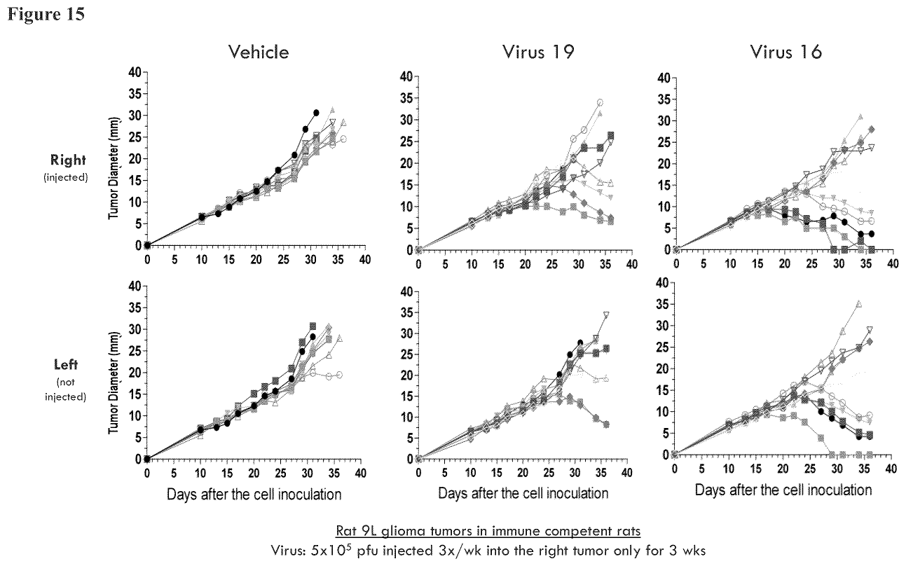

Effects on tumor growth were then observed for a further ˜30 days. This demonstrated superior tumor control and shrinkage with the virus expressing GALV-R- in both injected and uninjected tumors, demonstrating improved systemic effects. This is shown in FIG. 15. FIG. 10 shows that a virus expressing GALV (Virus 15) also shows enhanced killing of rat 91 cells in vitro as compared to a virus which does not express GALV (Virus 24).

### Example 11. A Virus of the Invention Modified for Oncolytic Use is Synergistic with Immune Checkpoint Blockade in Mouse Tumor Models

Virus 16 was tested in mice harboring CT26 tumors in the left and right flanks. One million tumor cells were first implanted in both flanks of Balb/c mice and tumors allowed to grow to 0.5-0.6 cm in diameter.

Groups of 10 mice were then treated with:
Vehicle (3 injections into right flank tumors every other day);
5×10exp6 pfu of Virus 16 injected in the right flank tumor every other day;
anti-mouse PD1 alone (10 mg/kg i.p. every three days, BioXCell clone RMP1-14);
anti-mouse CTLA-4 (3 mg/Kg i.p every three days, BioXCell clone 9D9);
anti-mouse PD1 together with Virus 16;
anti-mouse CTLA4 together with Virus 16;
1-methyl trypotophan (IDO inhibitor (5 mg/ml in drinking water));
anti-mouse PD1 together with 1-methyl trypotophan;
anti-mouse PD1 together with 1-methyl trypotophan and Virus 16;

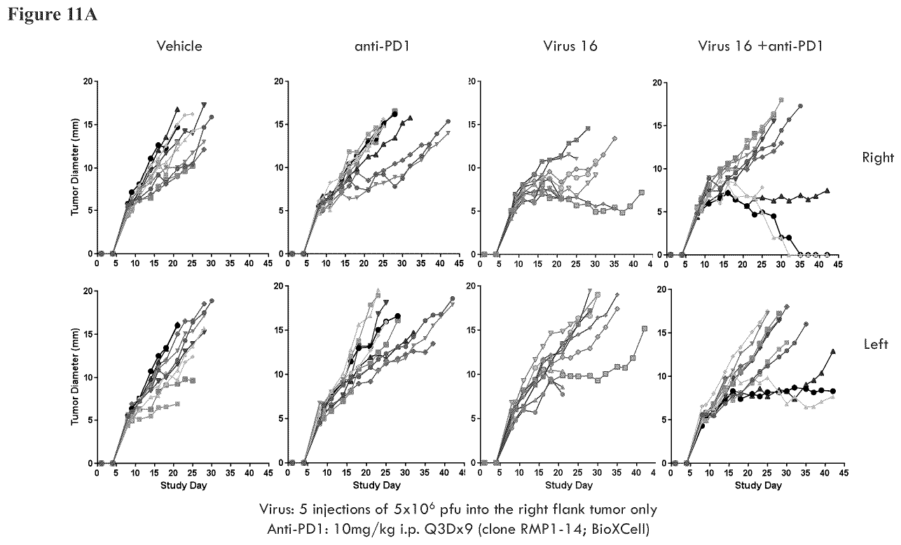

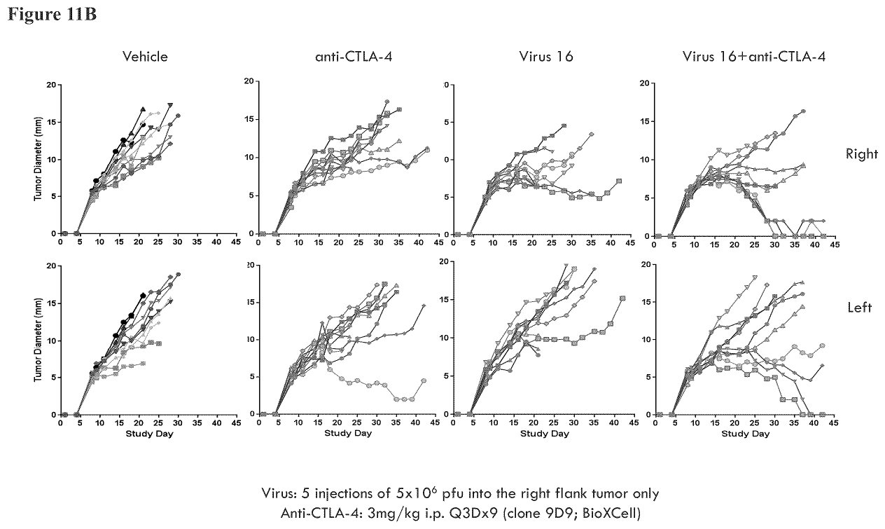

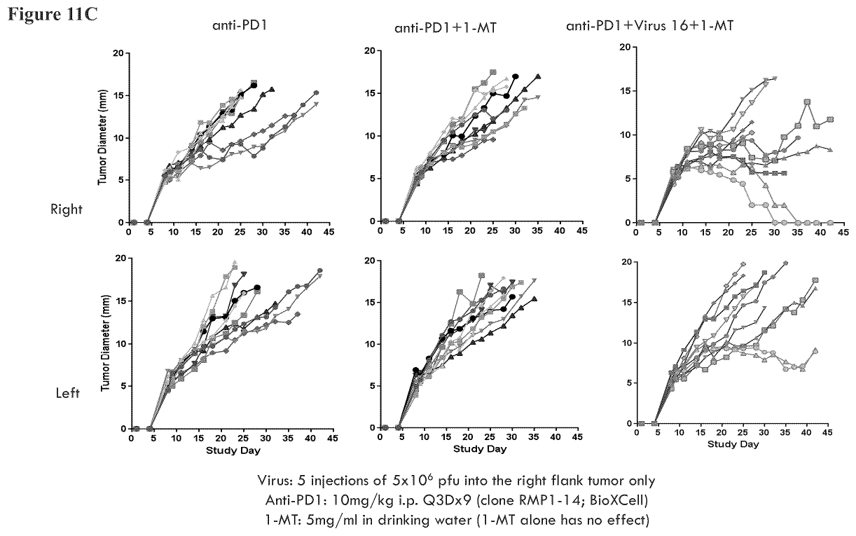

Effects on tumor size were observed for a further 30 days. A greater tumor reduction in animals treated with combinations of virus and checkpoint blockade was demonstrated than in animals treated with the single treatment groups (see FIGS. 11A-11C). Enhanced tumor reduction with Virus 16 together with both anti-PD1 and IDO inhibition was also demonstrated as compared to Virus 16 together with only anti-PD1 (see FIGS. 11A-11C).

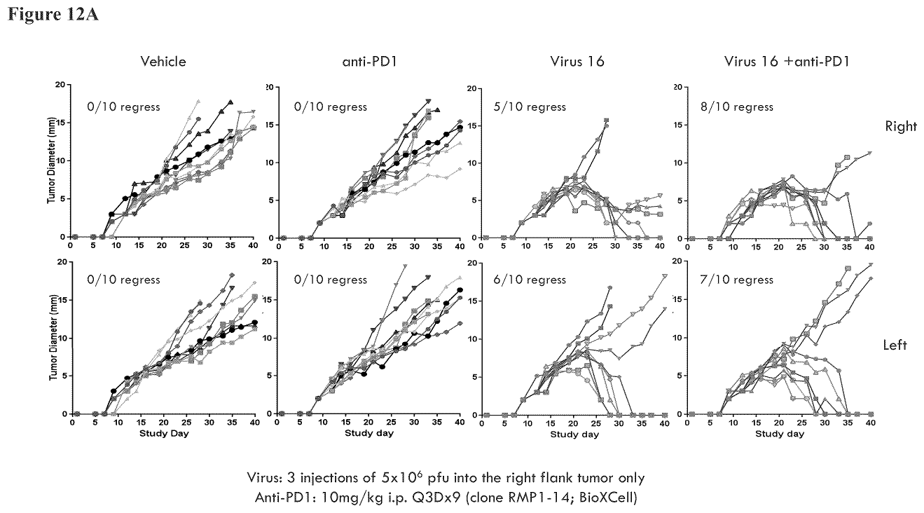

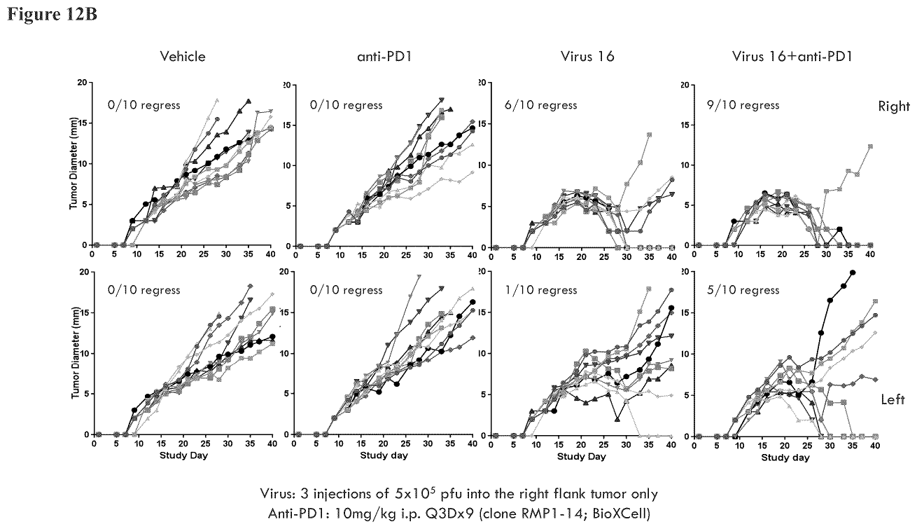

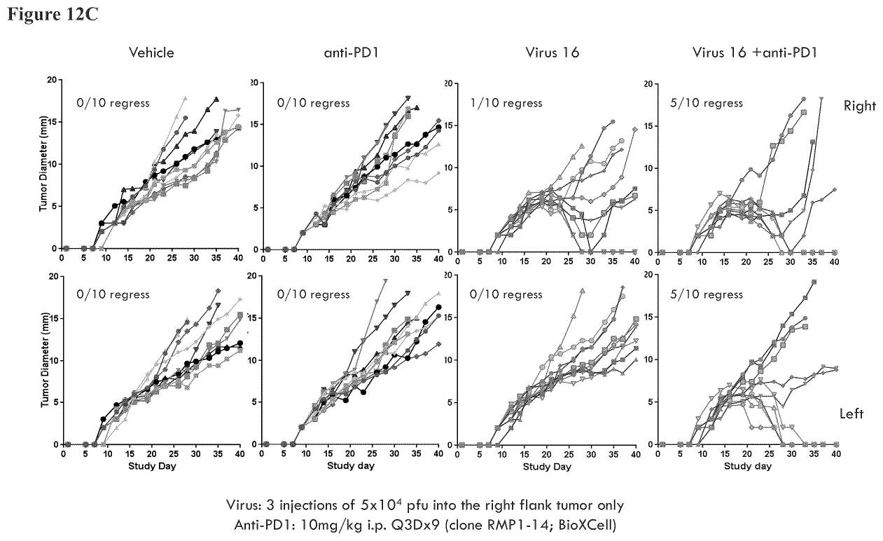

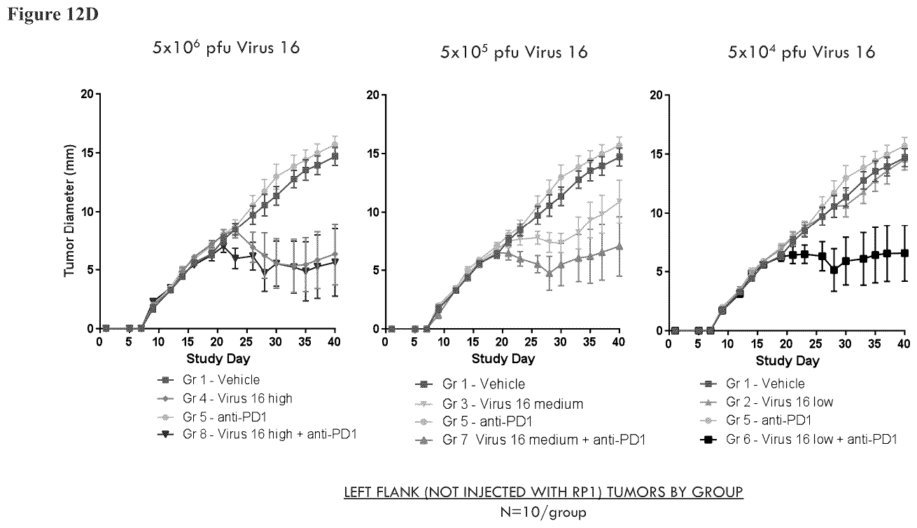

Enhanced activity of Virus 16 in combination with immune checkpoint blockade was also seen in A20 tumors (FIGS. 12A-12D).

### Example 12. The Effect of the Expression of a Fusogenic Protein from an Oncolytic Virus of the Invention in Human Xenograft Models in Immune Deficient Mice

The GALV R-protein causes cell to cell fusion in human cells but not in mouse cells. However, human xenograft tumors grown in immune deficient mice can be used to assess the effects of GALV expression on anti-tumor efficacy.

The utility of the invention was therefore further demonstrated by administering A549 human lung cancer cells into the flanks of nude mice and allowing the tumors to grow to approximately 0.5 cm in diameter.

The following treatments were then administered to groups of mice (ten per group), into tumor containing flank of each mouse three times over one week:
50 μl of vehicle;
50 μl of 10⁷ pfu/ml of Virus 16 (expresses both mouse GM-CSF and GALV-R-);
50 μl of 10⁶ pfu/ml of Virus 16;
50 μl of 10⁵ pfu/ml of Virus 16;
50 μl of 10⁷ pfu/ml of Virus 19 (expresses only mouse GM-CSF);
50 μl of 10⁶ pfu/ml of Virus 19;
50 μl of 10⁵ pfu/ml of Virus 19.

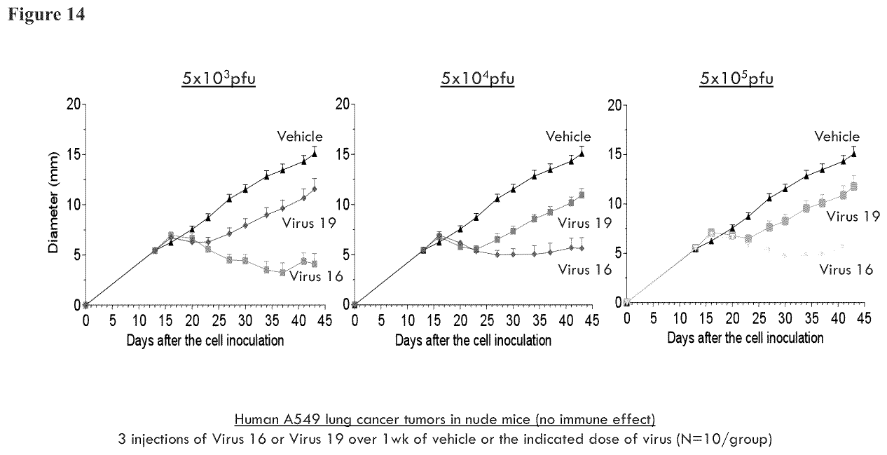

Effects on tumor growth were then observed for a further ≈30 days. This experiment demonstrated superior tumor control and shrinkage with the virus expressing GALV-R- in both tumor models (see FIG. 14).

### Example 13. Expression of Two Immune Stimulatory Molecules from a Virus Expressing a Fusogenic Protein

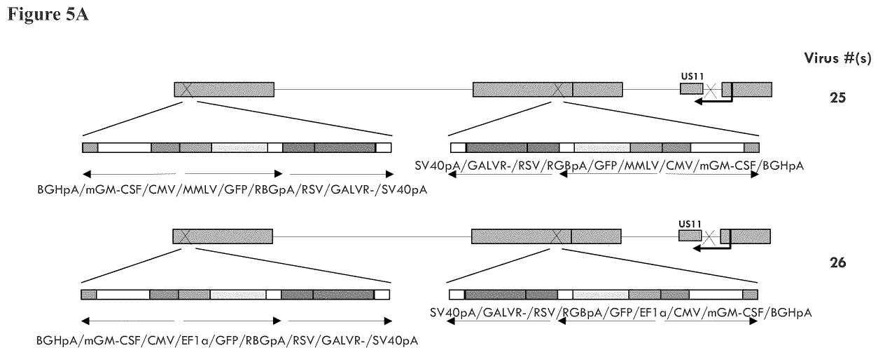

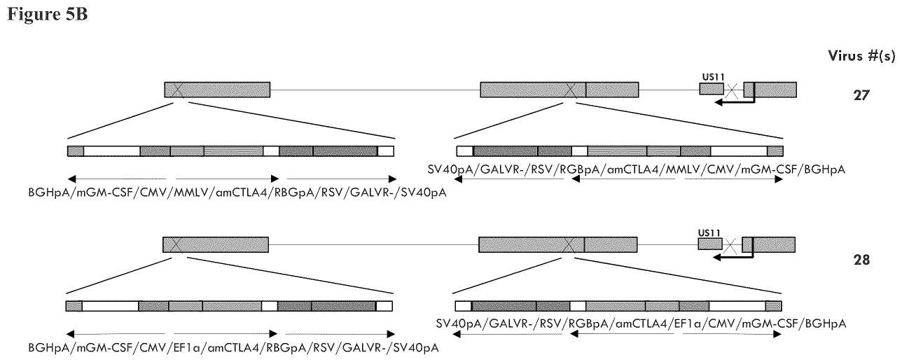

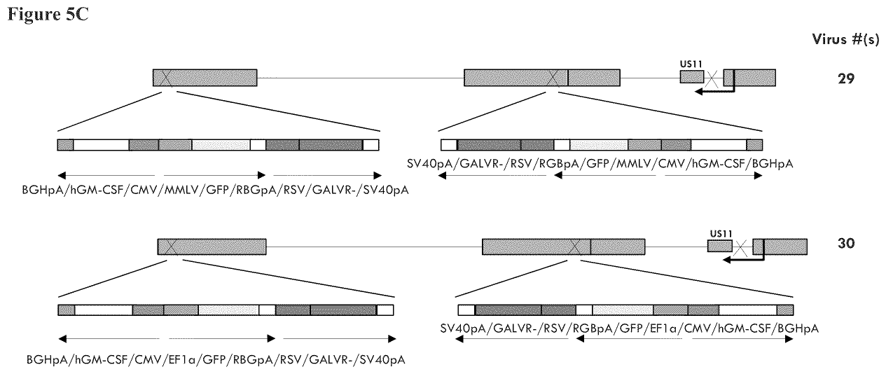

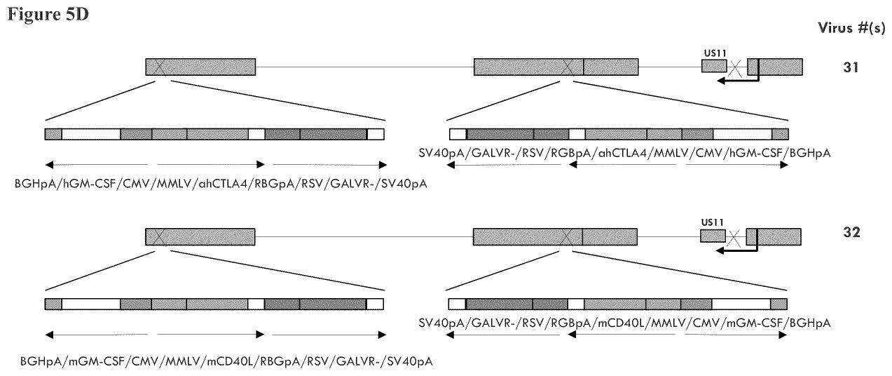

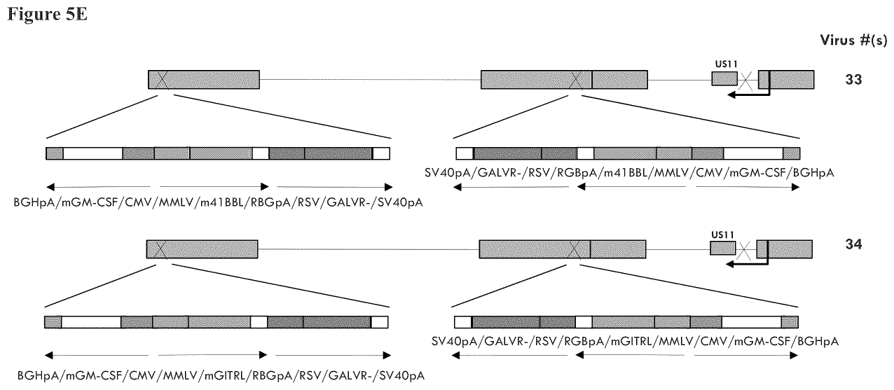

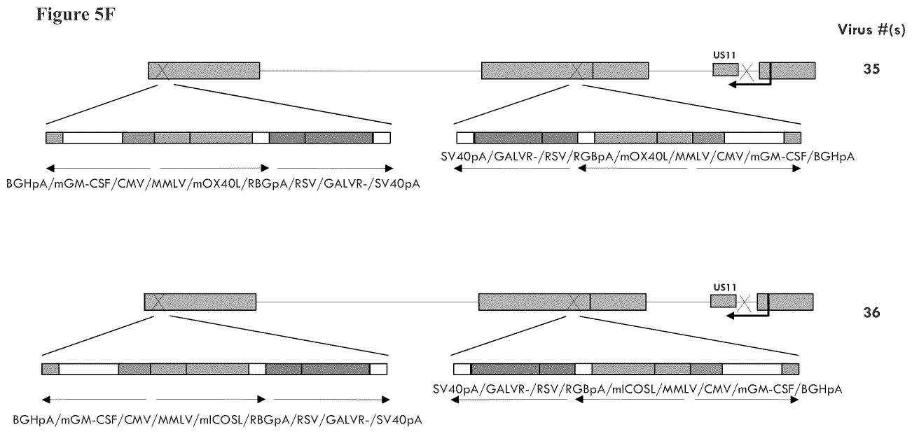

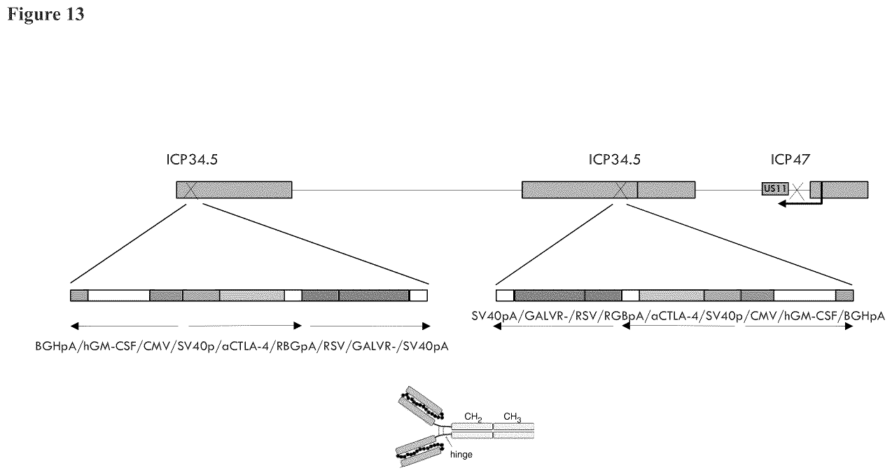

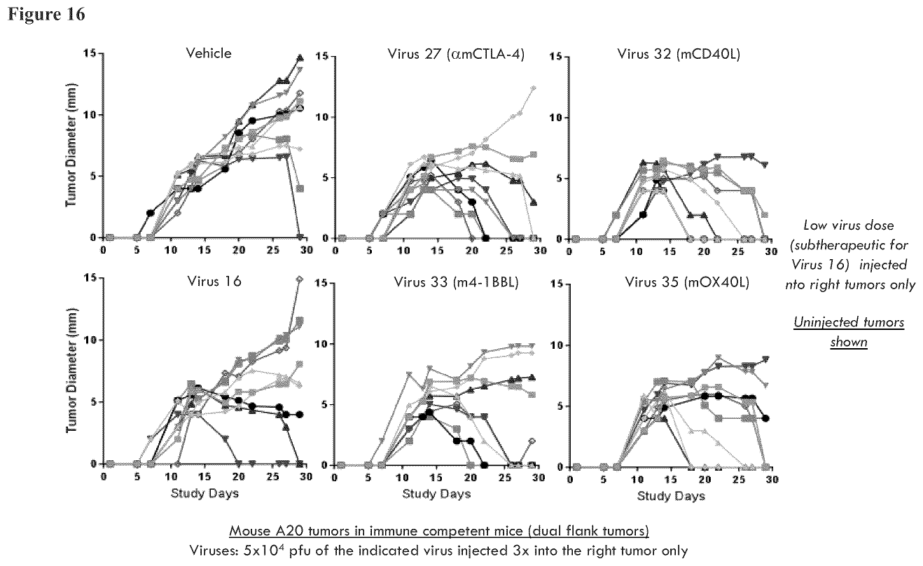

Viruses similar to the GALV-R- and mGM-CSF expressing virus described above (Virus 16) were constructed, but additionally expressing mouse versions of CD40L (virus 32), ICOSL (virus 36), OX40L (virus 35), 4-1BBL (virus 33) and GITRL (virus 34). Here, instead of using a plasmid containing ICP34.5 flanking regions and an expression cassette comprising GM-CSF and GALV-R-driven by a CMV and an RSV promoter, a plasmid containing ICP34.5 flanking regions and an expression cassette comprising GM-CSF, GALV and the additional proteins driven by a CMV, an RSV and an MMLV promoter respectively were used for recombination with a virus containing GM-CSF, GALV and GFP inserted into ICP34.5. Non-GFP expressing plaques were again selected. Correct insertion was confirmed by PCR, and expression by western blotting and/or ELISA for the additional inserted gene. These viruses are shown in FIGS. 5A-5F. Similarly, viruses expressing anti-mouse and anti-human CTLA-4 in addition to GALV and mGM-CSF were also constructed (Viruses 27 and 31 in FIGS. 5A-5F and see also FIG. 13). Effects of viruses expressing anti-mouse CTLA-4 (virus 27), mCD40L (virus 32), m4-1BBL (virus 33) or mOX40L (virus 35) in addition to mGM-CSF and GALVR- in vivo is shown in FIG. 16 which showed enhanced activity in A20 tumors as compared to virus 16 (expresses mGM-CSF and GALVR-). In these experiments tumors were induced in both flanks of mice, and virus or vehicle injected only into the right flank tumor. The dose of virus used was 5×10⁴ pfu (50 ul of 1×10⁶ pfu/ml in each case), given three times over one week. This dose level of virus is subtherapeutic for uninjected tumors for virus 16, which allows the benefits of the delivery of the additional molecules encoded by viruses 27, 32, 33 and 35 to clearly be seen.

### DEPOSIT INFORMATION

The following HSV1 strains were deposited at the ECACC, Culture Collections, Public Health England, Porton Down, Salisbury, SP4 OJG, United Kingdom on 19 Dec. 2016 by Replimune Limited and were allocated the indicated provisional accession numbers:
RH004A—Provisional Accession Number 16121902
RH015A—Provisional Accession Number 16121903
RH018A—Provisional Accession Number 16121904
RH021A—Provisional Accession Number 16121905
RH023A—Provisional Accession Number 16121906
RH031A—Provisional Accession Number 16121907
RH040B—Provisional Accession Number 16121908
RH047A—Provisional Accession Number 16121909.

1. An oncolytic virus comprising: (i) a GM-CSF-encoding gene; and (ii) an immune co-stimulatory pathway activating molecule or an immune co-stimulatory pathway activating molecule-encoding gene.

2. The virus of claim 1, wherein the immune co-stimulatory pathway activating molecule-encoding gene encodes CD40 ligand (CD40L), ICOS ligand, GITR ligand, 4-1-BB ligand, OX40 ligand, TL1A, CD30 ligand, CD27 or flt3 ligand or a modified version of any of these.

3. The virus of claim 1 or 2, wherein the immune co-stimulatory pathway activating molecule-encoding gene encodes CD40 ligand, GITR ligand, 4-1-BB ligand, OX40 ligand, ICOS ligand or a modified version of any of these.

4. The virus of claim 1, wherein the immune co-stimulatory pathway activating molecule-encoding gene encodes a CTLA-4 inhibitor.

5. The virus of claim 4, wherein the CTLA-4 inhibitor is a CTLA-4 antibody or fragment thereof.

6. The virus of any one of claims 1 to 5, further comprising a fusogenic protein-encoding gene.

7. The virus of claim 6 where the fusogenic protein is selected from the group consisting of vesicular stomatitis virus (VSV) G-protein, syncitin-1, syncitin-2, simian virus 5 (SV5) F-protein, measles virus (MV) H-protein, MV F-protein, respiratory syncytial virus (RSV) F-protein and a glycoprotein from gibbon ape leukemia virus (GALV), murine leukemia virus (MLV), Mason-Pfizer monkey virus (MPMV) or equine infectious anaemia virus (EIAV) from which the R peptide has been deleted.

8. The virus of claim 6 or 7, wherein the fusogenic protein is the glycoprotein from gibbon ape leukemia virus (GALV) and has the R transmembrane peptide mutated or removed (GALV-R-).

9. The virus of any one of the preceding claims, which encodes more than one immune co-stimulatory pathway activating molecule.

10. The virus of any one of the preceding claims, which is derived from a clinical isolate of a virus.

11. The virus of any one of the preceding claims, which is a modified clinical isolate of a virus, wherein the clinical isolate kills two or more tumor cell lines more rapidly and/or at a lower dose in vitro than one or more reference clinical isolates of the same species of virus.

12. The virus of claim 10 or 11, wherein the clinical isolate is
strain RH018A having the provisional accession number ECCAC 16121904;
strain RH004A having the provisional accession number ECCAC 16121902;
strain RH031A having the provisional accession number ECCAC 16121907;
strain RH040B having the provisional accession number ECCAC 16121908;
strain RH015A having the provisional accession number ECCAC 16121903;
strain RH021A having the provisional accession number ECCAC 16121905;
strain RH023A having the provisional accession number ECCAC 16121906; or
strain RH047A having the provisional accession number ECCAC 16121909.

13. The virus of any one of claims 1 to 11, which is selected from the group consisting of herpes viruses, pox viruses, adenoviruses, retroviruses, rhabdoviruses, paramyxoviruses and reoviruses.

14. The virus of any one of the preceding claims, which is a herpes simplex virus (HSV).

15. The virus of claim 14 which is a HSV1.

16. The virus of claim 15, wherein the HSV:
(a) does not express functional ICP34.5;
(b) does not express functional ICP47; and/or
(c) expresses the US11 gene as an immediate early gene.

17. The virus of any one of claims 14 to 16, wherein the GM-CSF-encoding gene and an immune co-stimulatory pathway activating molecule-encoding gene are inserted into the ICP34.5 encoding locus, either by insertion, or partial or complete deletion, in a back to back orientation in relation to each other, each under separate regulatory control.

18. The virus of any one of the preceding claims, wherein the sequence of a gene encoding GM-CSF and/or the sequence of the gene encoding an co-immune stimulatory pathway activating molecule is codon optimized so as to increase expression levels in target cells.

19. A virus which expresses three heterologous genes, wherein each of the three heterologous genes is driven by a different promoter selected from the CMV promoter, the RSV promoter, the SV40 promoter (SEQ ID) and a retroviral LTR promoter.

20. A virus according to any one of the preceding claims, which expresses three heterologous genes, wherein each of the three heterologous genes is driven by a different promoter selected from the CMV promoter, the RSV promoter, the SV40 promoter and a retroviral LTR promoter.

21. The virus of claim 19 or 20, which expresses four heterologous genes driven by each of the CMV promoter, the RSV promoter, the SV40 promoter and a retroviral LTR promoter, respectively.

22. The virus of any one of claims 19 to 21, where the retroviral LTR is from MMLV.

23. A virus which expresses three heterologous genes, wherein each of the three heterologous genes is terminated by a different poly adenylation sequence selected from the BGH, SV40, HGH and RBG poly adenylation sequences.

24. A virus according to any one of the preceding claims, which expresses three heterologous genes, wherein each of the three heterologous genes is terminated by a different poly adenylation sequence selected from the BGH, SV40, HGH and RBG poly adenylation sequences.

25. The virus of claim 23 or 24, which expresses four heterologous genes terminated by each of the BGH, SV40, HGH and RBG poly adenylation sequences, respectively.

26. The virus of any one of claims 19 to 25 which is
(a) a HSV;
(b) a HSV1; or
(c) a pox virus.

27. A pharmaceutical composition comprising a virus according to any one of claims 1 to 26 and a pharmaceutically acceptable carrier or diluent.

28. The virus of any one of claims 1 to 26 for use in a method of treating the human or animal body by therapy.

29. The virus of any one of claims 1 to 26 for use in a method of treating cancer.

30. The virus for use according to claim 29, wherein the method comprises administering a further anti-cancer agent.

31. The virus for use according to claim 30, wherein the further anti-cancer agent is selected from an agent targeting an immune co-inhibitory or immune co-stimulatory pathway, radiation and/or chemotherapy, an agent that targets a specific genetic mutation which occurs in tumors, an agent intended to induce an immune response to one or more tumor antigen(s) or neoantigen(s), a cellular product derived from T cells or NK cells, an agent intended to stimulate the STING, cGAS, TLR or other innate immune response and/or inflammatory pathway, a second virus optionally an oncolytic virus, and combinations thereof.

32. The virus for use according to claim 30 or 31, wherein the agent targeting an immune co-inhibitory pathway is a CTLA-4 inhibitor, a PD-1 inhibitor, a PD-L1 inhibitor, a LAG-3 inhibitor, a TIM-3 inhibitor, a VISTA inhibitor, aCSF1R inhibitor, an IDO inhibitor, a KIR inhibitor, a SLAMF7 inhibitor, a CEACAM1 inhibitor or a CD47 inhibitor, and/or the agent targeting an immune co-stimulatory pathway is a GITR agonist, a 4-1-BB agonist, an OX40 agonist, a CD40 agonist or an ICOS agonist.

33. The virus for use according to any one of claims 30 to 32, wherein the further anti-cancer agent is an antibody.

34. The virus for use according to any one of claims 30 to 33, wherein the method comprises administering an inhibitor of the indoleamine 2,3-dioxygenase (IDO) pathway and a further antagonist of an immune co-inhibitory pathway, or an agonist of an immune co-stimulatory pathway.

35. The virus for use according to any one of claims 29 to 34, wherein the virus and the further anti-cancer agent(s) are administered separately.

36. The virus for use according to any one of claims 29 to 34, wherein the virus and the further anti-cancer agent(s) are administered concurrently.

37. The virus for use according to any one of claims 29 to 36, wherein the cancer is a solid tumor.

38. A product of manufacture comprising a virus according to any one of claims 1 to 26 in a sterile vial, ampoule or syringe.

39. A method of treating cancer, which comprises administering a therapeutically effective amount of the virus of any one of claims 1 to 26 or a pharmaceutical composition according to claim 27 to a patient in need thereof.

40. A method according to claim 39, which further comprises administering a therapeutically effective amount of a further anti-cancer agent to a patient in need thereof.

41. A method according to claim 40, wherein the further anti-cancer agent is selected from the group consisting of an agent targeting an immune co-inhibitory or immune co-stimulatory pathway, radiation and/or chemotherapy, an agent that targets a specific genetic mutation which occurs in tumors, an agent intended to induce an immune response to one or more tumor antigen(s) or neoantigen(s), a cellular product derived from T cells or NK cells, an agent intended to stimulate the STING, cGAS, TLR or other innate immune response and/or inflammatory pathway, a second virus optionally an oncolytic virus, and combinations thereof.

42. A method according to claim 41, wherein the agent targeting an immune co-inhibitory pathway is a CTLA-4 inhibitor, a PD-1 inhibitor, a PD-L1 inhibitor, a LAG-3 inhibitor, a TIM-3 inhibitor, a VISTA inhibitor, aCSF1R inhibitor, an IDO inhibitor, a KIR inhibitor, a SLAMF7 inhibitor, a CEACAM1 inhibitor or a CD47 inhibitor, and/or the agent targeting an immune co-stimulatory pathway is a GITR agonist, a 4-1-BB agonist, an OX40 agonist, a CD40 agonist or an ICOS agonist.

43. A method according to claim 41 or 42, wherein the further anti-cancer agent comprises an antibody.

44. A method according to any one of claims 40 to 43, wherein the virus and the further anti-cancer agent(s) are administered separately.

45. A method according to any one of claims 40 to 43, wherein the virus and the further anti-cancer agent(s) are administered concurrently.

46. A method according to any one of claims 40 to 45, wherein the cancer is a solid tumor.

47. Use of the virus of any one of claims 1 to 26 in the manufacture of a medicament for use in a method of treating cancer.

48. Use according to claim 47, wherein the method comprises administering a further anti-cancer agent.
